(19) United States
(12) Patent Application Publication
(10) Pub. No.: US 2022/0196520 A1
(43) Pub. Date: Jun. 23, 2022

(54) CELL RECOVERY APPARATUS, CELL RECOVERY METHOD, AND COMPUTER READABLE MEDIUM

(71) Applicant: OLYMPUS CORPORATION, Tokyo (JP)

(72) Inventors: Takuma DEZAWA, Tokyo (JP); Yoko KAMATO, Tokyo (JP); Elena SHIMOJI, Tokyo (JP)

(73) Assignee: OLYMPUS CORPORATION, Tokyo (JP)

(21) Appl. No.: 17/529,426

(22) Filed: Nov. 18, 2021

(30) Foreign Application Priority Data
Dec. 17, 2020 (JP) 2020-209371

(51) Int. Cl. G01N 1/14; G06K 9/00; C12Q 3/00; B01L 3/00; G01N 15/10

(52) U.S. Cl. CPC G01N 1/14; G01N 2001/1012; C12Q 3/00; B01L 3/561; G01N 15/1056; G01N 2015/1006; G01N 2015/1081; B01L 2200/025; B01L 2200/026; B01L 2200/0647; B01L 2200/143; B01L 2300/023; B01L 2300/027; B01L 2300/0663; B01L 2400/049; G06K 9/00134

(57) ABSTRACT

A cell recovery apparatus includes an observation apparatus for observing the inside of a medium in a container via an insertion section to be inserted into the medium, and a recovery apparatus that is positioned relative to a predetermined region to be observed by the observation apparatus and recovers a cell in the medium from the container.

### CROSS REFERENCE TO RELATED APPLICATIONS

This application is based upon and claims the benefit of priority of the prior Japanese Patent Application No. 2020-209371, filed Dec. 17, 2020, the entire contents of which are incorporated herein by reference.

### BACKGROUND OF THE INVENTION

### Field of the Invention

The present discloser relates to a cell recovery apparatus that recovers cultured cells, a cell recovery method, and a computer readable medium.

### Description of the Related Art

For popularization of drug development and regenerative medicine using pluripotent stem cells, a culture technique for stably supplying a large number of cells while maintaining a predetermined level or more of quality is indispensable. Accordingly, in recent years, suspension culture that allows a large number of cells to be cultured at one time has drawn more attention than monolayer culture.

When cultured cells are clinically applied, the cultured cells need to be sorted. To sort the cells, a cell sorter, for example, can be used. A technique for sorting cells without using a cell sorter has also been proposed. Japanese Patent Laid-Open No. 2017-108738, for example, discloses that cells guided to a predetermined position by dielectrophoresis are adhered and held, that the target cell is detected from respective images of the held cells, and that the detected target cell is recovered. The cells can be sorted even by using a technique described in Japanese Patent Laid-Open No. 2017-108738.

### SUMMARY OF THE INVENTION

A cell recovery apparatus according to an aspect of the present invention includes an observation apparatus includes an insertion section to be inserted into a medium in a container, the observation apparatus being for observing the inside of the medium via the insertion section, and a recovery apparatus that is positioned relative to a predetermined region to be observed by the observation apparatus and recovers a cell in the medium from the container.

A cell recovery method according to an aspect of the present invention includes imaging a predetermined region in a medium in a container with an insertion section in an imaging apparatus inserted into the medium, and recovering a cell in the medium from the container by a recovery apparatus positioned relative to the predetermined region in response to a result of object detection for an image acquired by the imaging apparatus.

A non-transitory computer readable medium according to an aspect of the present invention stores a program for causing a computer to perform processing for performing object detection for an image acquired by an imaging apparatus imaging a predetermined region in a medium in a container with an insertion section in the imaging apparatus inserted into the medium, and controlling a recovery apparatus that is positioned relative to the predetermined region and recovers a cell in the medium from the container in response to a result of the object detection.

### BRIEF DESCRIPTION OF THE DRAWINGS

The present invention will be more apparent from the following detailed description when the accompanying drawings are referenced.

FIG. 1 is a schematic configuration diagram for describing an entire configuration of a cell recovery apparatus 1;

FIG. 2 is a diagram illustrating a configuration of a cell recovery apparatus 100 according to a first embodiment;

FIG. 3 is a block diagram illustrating a functional confirmation of the cell recovery apparatus 100;

FIG. 4 is a flowchart illustrating an example of cell recovery processing to be performed by the cell recovery apparatus 100;

FIG. 5 is a flowchart illustrating an example of suction control processing to be performed by the cell recovery apparatus 100;

FIG. 6 is a flowchart illustrating another example of suction control processing to be performed by the cell recovery apparatus 100;

FIG. 7 is a flowchart illustrating still another example of suction control processing to be performed by the cell recovery apparatus 100;

FIG. 8 is a diagram illustrating a configuration of a stereo optical system 150;

FIG. 9 is a flowchart illustrating still another example of cell recovery processing to be performed by the cell recovery apparatus 100;

FIG. 10 is a diagram illustrating a configuration of a recovery nozzle 124;

FIG. 11 is a diagram illustrating a configuration of a recovery nozzle 126;

FIG. 12 is a diagram illustrating a configuration of a cell recovery apparatus 200 according to a second embodiment;

FIG. 13 is a diagram for describing a modification of the cell recovery apparatus 200;

FIG. 14 is a diagram illustrating a configuration of an insertion section 230;

FIG. 15 is a diagram illustrating a configuration of an insertion section 240;

FIG. 16 is a diagram illustrating a configuration of an insertion section 250;

FIG. 17 is a diagram illustrating a configuration of a cell recovery apparatus 300 according to a third embodiment;

FIG. 18 is a flowchart illustrating an example of cell recovery processing to be performed by a cell recovery apparatus according to a fourth embodiment;

FIG. 19 is a diagram illustrating a configuration of an insertion section 411;

FIG. 20 is a diagram for describing an example of height control of the insertion section 411;

FIG. 21 is a flowchart illustrating an example of cell recovery processing to be performed by a cell recovery apparatus according to a fifth embodiment;

FIG. 22 is a diagram illustrating an example of an image to be displayed on a display apparatus 133;

FIG. 23 is a flowchart illustrating an example of cell recovery processing to be performed by a cell recovery apparatus according to a sixth embodiment;

FIG. 24 is a diagram illustrating another example of an image to be displayed on a display apparatus 133;

FIG. 25 is a diagram illustrating still another example of an image to be displayed on the display apparatus 133;

FIG. 26 is a diagram illustrating still another example of an image to be displayed on the display apparatus 133;

FIG. 27 is a diagram illustrating still another example of an image to be displayed on the display apparatus 133;

FIG. 28 is a diagram illustrating still another example of an image to be displayed on the display apparatus 133;

FIG. 29 is a diagram for describing an operation of an extension nozzle 321a;

FIG. 30 is a diagram illustrating a configuration of a cell recovery apparatus 600 according to a seventh embodiment; and

FIG. 31 is a diagram illustrating a configuration of a cell recovery apparatus 700 according to an eighth embodiment.

### DESCRIPTION OF THE EMBODIMENTS

Living cells are very delicate and are susceptible to a damage. Accordingly, a stress on the cells at the time of cell recovery is desirably suppressed as much as possible. Particularly when a clinical application is assumed, this point is very important.

However, cell sorting using a cell sorter is not generally desirable when a clinical application is presupposed because an SICS (sorter-induced cellular stress) occurs. Even in cell sorting using a technique described in Japanese Patent Laid-Open No. 2017-108738, a procedure for transferring cells once to an environment different from a culture environment and then recovering the target cell is taken, whereby the cells are stressed during complicated recovery work.

Thus, in all the above-described cell sorting techniques, it is concerned that cells that are required to be delicately handled are adversely affected. Accordingly, a new technique for sorting cultured cells has been required.

Embodiments of the present invention will be described below.

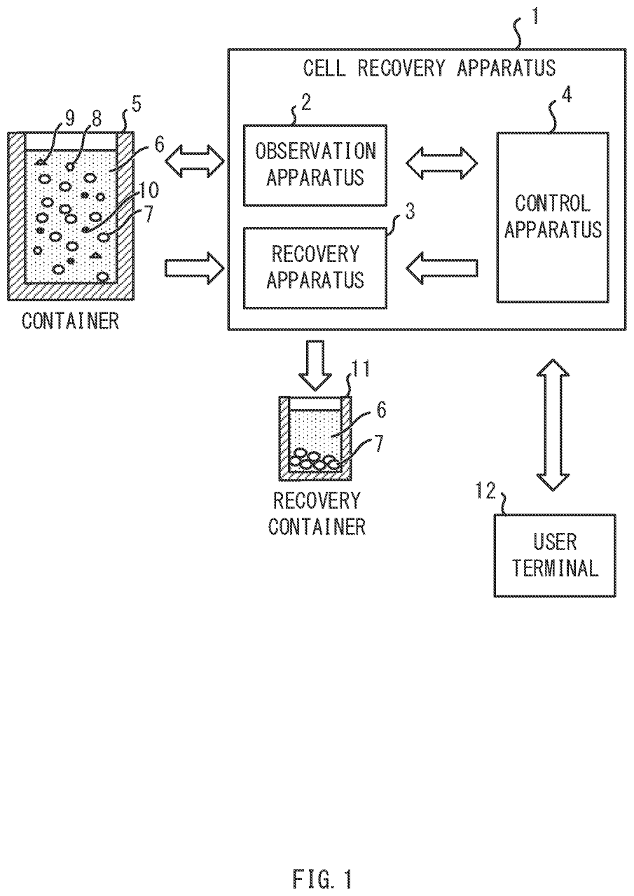

FIG. 1 is a schematic configuration diagram for describing an entire configuration of a cell recovery apparatus 1 according to an embodiment. The cell recovery apparatus 1 includes an observation apparatus 2 for observing the inside of a medium 6 accommodated in a container 5 and a recovery apparatus 3 that recovers cells in the medium 6 from the container 5. The cell recovery apparatus 1 is an apparatus that sorts the cells in the medium 6, and the cell (e.g., a cell 7) to be recovered is discharged into a recovery container 11. The cell recovery apparatus 1 may further include a control apparatus 4, as illustrated in FIG. 1. The cell recovery apparatus 1 may not include the control apparatus 4. The cell recovery apparatus 1 may communicate with a user terminal 12, or may perform cell recovery in response to an instruction from the user terminal 12. The cell recovery apparatus 1 may notify the user terminal 12 of an elapse of recovery, a recovery result, and the like.

A cell recovery apparatus according to each of embodiments, described below, will be outlined with reference to FIG. 1. Although an example in which the cell recovery apparatus 1 recovers the cells cultured in suspension culture is illustrated in FIG. 1, a method of culturing the cell to be recovered by the cell recovery apparatus 1 is not limited to the suspension culture. Although the suspension culture is desirable in that a large number of cells can be more efficiently cultured than two-dimensional culture such as monolayer culture or rotary culture in which a proliferation force depends on the surface area of a container, the cell recovery apparatus 1 may be used to recover cells cultured in the two-dimensional culture. The cell recovery apparatus 1 may be used to recover cells cultured in any three-dimensional culture.

The suspension culture includes culture for suspending adherent cells, together with carriers such as microbeads, in a culture medium with the adherent cells respectively adhering to the carriers and proliferating the adherent cells in addition to general suspension culture for proliferating floating cells with the floating cells suspended in a liquid culture medium, for example. That is, the suspension culture in this specification means a method of culturing the cells with the cells suspended in the culture medium regardless of whether or not the cells respectively adhere to the carriers, unless otherwise specified.

The medium 6 accommodated in the container 5 is a culture medium, for example, and may be a liquid culture medium, for example. When cells are cultured in culture other than the suspension culture, such as three-dimensional culture, for example, the medium 6 may be a solid culture medium such as gels. The medium 6 may not be a culture medium.

The container 5 is a container in which cells, together with the medium 6, are accommodated. The container 5 may be a culture container, for example. In the case, the cells may be cultured in the container 5. That is, the cell recovery apparatus 1 may be an apparatus that directly recovers the cells from the container 5 as the culture container. The culture container may be a relatively small-sized container such as a spinner flask or a culture bag, or may be a container being of a relatively large size and having a complicated structure, such as a bioreactor.

The container 5 may be a general container other than the culture container, for example. Cells, together with a culture medium, may be transferred to the container 5 from the culture container, and the cell recovery apparatus 1 may transfer the cells to the container 5 from the culture container and then recover the cells from the container 5. Cells may be transferred to the container 5 accommodating a medium, which is not a culture medium, from the culture container, and the cell recovery apparatus 1 may transfer the cells to the container 5 accommodating a medium other than a culture medium and then recover the cells from the container 5. That is, the cell recovery apparatus 1 may be an apparatus that recovers cells from the container 5 in an environment different from a culture environment.

The recovery container 11 is a container that accommodates cells recovered by the recovery apparatus 3. The recovery container 11 may be a culture container, for example. In the case, the cells recovered by the recovery apparatus 3 may further be cultured in the container 11. Although an example in which the recovery container 11 is not included in the cell recovery apparatus 1 is illustrated in FIG. 1, the recovery container 11 may constitute the recovery apparatus 3.

The observation apparatus 2 is an apparatus for observing the inside of the medium 6 accommodated in the container 5. The observation apparatus 2 is an apparatus for optically observing the inside of the medium 6, and may form an optical image of a region in the medium 6. The observation apparatus 2 may include an imaging apparatus, and may be a microscope apparatus including a digital camera, an endoscope, and a digital camera, for example. The observation apparatus 2 may project an optical image onto the eye of an observer or a screen, and may be a microscope apparatus including an eyepiece or a projector, for example.

The observation apparatus 2 is desirably an observation apparatus for observing the inside of the medium 6 with at least a distal end portion of the observation apparatus 2 inserted into the medium 6. That is, the observation apparatus 2 includes an insertion section to be inserted into the medium 6 in the container 5, and desirably allows observation of the inside of the medium 6 via the insertion section. This is because when the inside of the medium 6 is observed via the insertion section inserted into the medium 6, an observation performance does not depend on a shape, transparency (a refractive index), and the like of the container 5, in contrast to when the inside of the medium 6 is observed via the container 5. Therefore, the observation apparatus 2 allows observation of the inside of the medium 6 accommodated in any container 5.

The observation apparatus 2 may have a sufficient resolution to observe the cells in the medium 6, and can desirably identify various types of cells (the cell 7, a cell 8, and cell 9, and a cell 10) existing in the container 5, a cell mutation, a differentiated state, and the like. The observation apparatus 2 may be able to visualize the cells as phase objects, and an observation method is not particularly limited. The observation apparatus 2 may visualize the cells in the medium 6 using a bright field observation method, for example, or may visualize the cells using other observation methods suitable for observation of the phase objects, such as a phase difference observation method. Considering a clinical application of cells, the observation method is desirably an observation method with which the cells can be observed with the cells unstained. The observation apparatus 2 may visualize cells using a fluorescence observation method. In this case, the cells may be labeled using a fluorescent dye or the like. A wavelength area of observation light is not particularly limited. The observation light may be visible light or infrared light.

The recovery apparatus 3 is an apparatus that recovers cells in the medium 6 from the container 5. Although a method of recovering the cells by the recovery apparatus 3 is suction using a suction nozzle, for example, a method for recovery by the recovery apparatus 3 is not particularly limited. The recovery apparatus 3 may recover cells by extruding the cells out of the container 5 using air feeding or liquid feeding from the nozzle instead of the suction, for example. The recovery apparatus 3 may recover cells by scooping the cells out of the container 5.

The recovery apparatus 3 may recover necessary cells in a subsequent process, or may recover unnecessary cells to remove the unnecessary cells from the container 5. That is, in the recovery apparatus 3, positive sorting for selectively recovering differentiated specific cells may be performed, or negative sorting for removing undifferentiated cells that may become cancerous may be performed, for example. The cell 7 to be recovered, which has been recovered by the recovery apparatus 3, is outputted to the recovery container 11, for example. The recovery apparatus 3 may recover the cell 7, together with the medium 6, or may output the cell 7, together with the medium 6, to the recovery container 11, as illustrated in FIG. 1.

The recovery apparatus 3 may be positioned relative to a predetermined region to be observed by the observation apparatus 2 (hereinafter referred to as an observation region). “The recovery apparatus 3 is positioned relative to the predetermined region” means that a region in the medium 6 where cells can be recovered using a recovery operation to be performed by the recovery apparatus 3 (hereinafter referred to as a recovery region) is positioned relative to the observation region, and a positional relationship at the time of at least cell recovery has been known between the observation region and the recovery region. If the positional relationship between the observation region and the recovery region has been known, a timing at which the recovery operation is to be performed to recover the cells from the recovery region can be grasped based on information obtained by observing the observation region. As a result, the recovery apparatus 3 can recover the cells depending on an observation result, that is, sort the cells.

The positional relationship between the observation region and the recovery region may be specified by observing the observation region, and may have been known as a result. For example, the recovery apparatus 3 may be positioned such that a distal end of the suction nozzle is observed, to position the recovery region relative to the observation region.

The positional relationship at the time of at least cell recovery may be known between the observation region and the recovery region. Therefore, the observation region and the recovery region need not match each other, and may not overlap each other. Assuming a case where a user who has observed the inside of the medium 6 using the observation apparatus 2 manually operates the recovery apparatus 3 so that the cells are recovered from the medium 6, the observation region and the recovery region are desirably regions at least respective parts of which overlap each other. If the observation region and the recovery region overlap each other, the user can recover, when finding out the cell to be recovered in the observation region, the cell by immediately operating the recovery apparatus 3. Accordingly, an operation of the cell recovery apparatus 1 is easy, and the user can appropriately recover the cells even if there is no support for a suction timing, for example, by the control apparatus 4.

The positional relationship at the time of at least cell recovery may be known between the observation region and the recovery region. The relationship may not be fixed. That is, a position and a size of each of the observation region and the recovery region may change depending on setting of each of the apparatuses. For example, the position and the size of the observation region may change depending on the setting of the observation apparatus 2, such as an observation magnification and an illumination condition. The position and the size of the recovery region may change depending on the setting of the recovery apparatus 3, such as a suction force.

The control apparatus 4 is a computer including at least a processor and a memory. The control apparatus 4 may be a general-purpose apparatus such as a personal computer, or may be a computer dedicated to the cell recovery apparatus 1. The control apparatus 4 may include a display device or an input device not illustrated, and may further include a communication device that communicates with the user terminal 12 or the like. The user terminal 12 is a terminal to be used by the user, for example, and is a notebook computer, a laptop computer, a tablet computer, or a smartphone, for example, although not particularly limited thereto.

The control apparatus 4 may be a recovery control apparatus that controls the recovery apparatus 3. The control apparatus 4 may analyze an image of the observation region acquired using an imaging apparatus included in the observation apparatus 2, to control the recovery apparatus 3 based on a result of the analysis for the image, for example. More specifically, the control apparatus 4 may control the recovery apparatus 3 based on a result of object detection for the image acquired using the imaging apparatus. The object detection may use a learned model obtained by deep layer learning. In the case, the learned model desirably learns at least a cell to be recovered. The learned model further desirably learns cells other than the cell to be recovered and objects other than the cells. The object detection may be performed using a learned model obtained by machine learning other than the deep layer learning, or an algorithm for detecting the cell to be recovered may be used for the object detection based on known feature values such as a shape and a size of the cell to be recovered. Specific control of the recovery apparatus 3 based on a result of the object detection is not particularly limited as long as the cell to be recovered can be recovered.

When the control apparatus 4 functions as the recovery control apparatus, a cell recovery process can be automated. Accordingly, cell culture can be labor-saved. When at least a part of a cell culture process including the cell recovery process is performed by remote control using the control apparatus 4 (and the user terminal 12), a chance for a person to enter an aseptic environment such as a culture chamber in which a culture container is placed can be reduced. Accordingly, a risk of contamination can also be reduced. These advantages are particularly beneficial in mass culture of cells in which a range of influence of contamination is large and an amount of work is enormous.

If the recovery apparatus 3 is directly operated by the user, the control apparatus 4 may not control the recovery apparatus 3. The control apparatus 4 may display the image of the observation region acquired using the imaging apparatus on a display section instead of controlling the recovery apparatus 3. The display section may be a part of the control apparatus 4 or an apparatus different from the control apparatus 4, and may be the user terminal 12, for example. In this case, the control apparatus 4 may further perform object detection for the image acquired using the imaging apparatus, or may notify the user of the presence of cells in the observation region based on a result of the object detection for the image. The control apparatus 4 may notify the user of the presence of cells by displaying additional information on the image of the observation region or may notify the presence of cells using other methods such as audio output, light emission, and vibration, for example.

The cell recovery apparatus 1 configured as described above makes it possible to selectively recover the desired cell, i.e., sort the cells by including the recovery apparatus 3, together with the observation apparatus 2 for observing the inside of the medium 6. As a result, erroneous cells and foreign substances can be avoided being mixed into subsequent processes, for example.

In the cell recovery apparatus 1, a centrifuge, a cell sorter, or the like need not be used to sort cells. Accordingly, cell recovery work can be more simplified, as compared with that in the conventional technique. Cells can also be selectively recovered while continuing to be cultured, or a culture process and a recovery process can be temporarily performed in parallel. Therefore, work efficiency of the entire cell culture can be enhanced. This point is particularly favorable for mass culture of cells the work efficiency of which is particularly emphasized.

In the cell recovery apparatus 1, cells can be directly sorted from the culture container. Accordingly, a damage to be received when equipment such as a centrifuge or a cell sorter is used and a damage by using a medical agent for detaching cells adhering to the container can be avoided. Further, a damage to cells caused by converging the cells to each equipment can also be avoided. Particularly, when the cell recovery apparatus 1 is applied to recovery of cells cultured in suspension culture, suspended cells can be recovered with a minimum of suction force. Accordingly, a stress to be received by the cells can be reduced without an excessively large force being applied to the cells at the time of recovery. Therefore, the quality of the recovered cells are easy to maintain, and the cells having a predetermined level or more of quality can be stably supplied.

The cell recovery apparatus according to each of embodiments will be specifically described below with reference to the drawings.

### First Embodiment

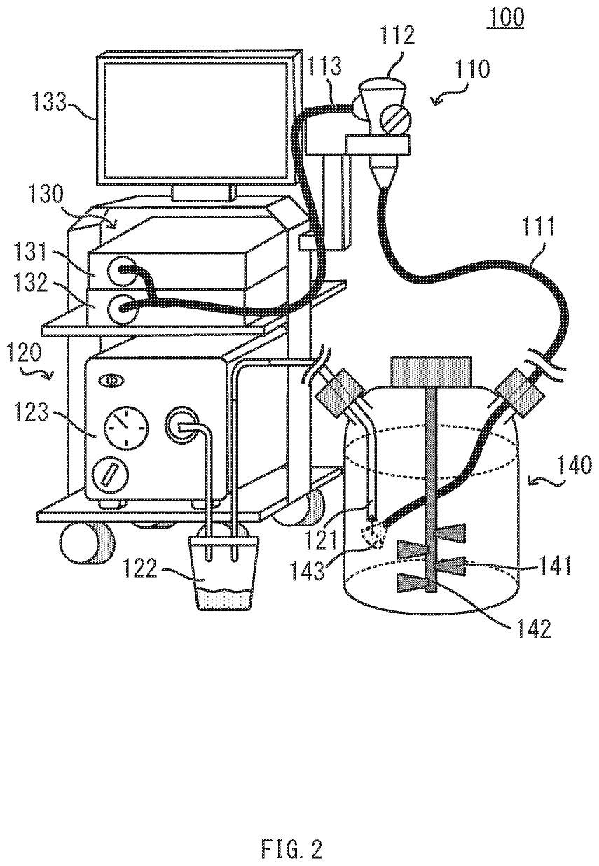

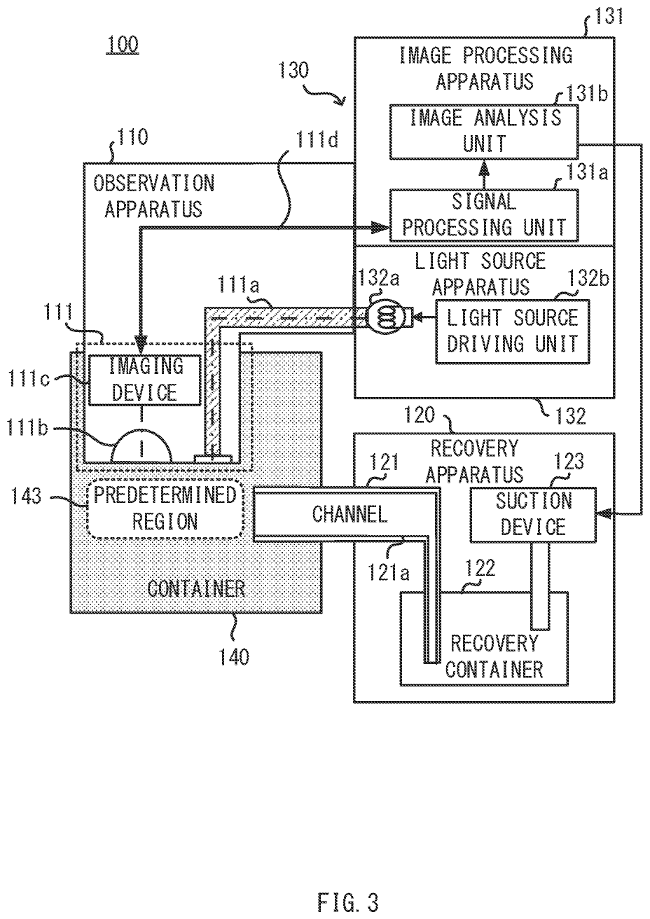

FIG. 2 is a diagram illustrating a configuration of a cell recovery apparatus 100 according to the present embodiment. FIG. 3 is a block diagram illustrating a functional confirmation of the cell recovery apparatus 100. The cell recovery apparatus 100 is an apparatus that automatically sorts cells cultured in suspension culture. First, a configuration of the cell recovery apparatus 100 will be described with reference to FIG. 2 and FIG. 3.

The cell recovery apparatus 100 includes an observation apparatus 110 for observing a predetermined region 143 in a medium accommodated in a container 140, a recovery apparatus 120 that recovers cells in the medium from the container 140, and a control apparatus 130 that controls the recovery apparatus 120, as illustrated in FIG. 2.

The container 140 is a spinner flask, for example, and cells are cultured in a liquid culture medium accommodated in the container 140. When a rotation shaft 142 in the container 140 rotates, a stirring blade 141 fixed to the rotation shaft 142 stirs the liquid culture medium. As a result, the cells are cultured with the cells suspended in the culture medium.

The observation apparatus 110 includes an insertion section 111 to be inserted into the culture medium in the container 140, an operation section 112 to be directly operated by a user, and a universal cord section 113 to be connected to the control apparatus 130, as illustrated in FIG. 2. The insertion section 111 can desirably fix a position of its distal end portion. The insertion section 111 may be hard, like an insertion section in a rigid endoscope, or may have a freely bendable structure by operating the operation section 112, like an insertion section in a flexible endoscope, for example.

The insertion section 111 is provided with a light guide 111a, an imaging optical system 111b, and an image sensor 111c, as illustrated in FIG. 3. The light guide 111a is an example of an illumination section that irradiates the predetermined region 143 in the culture medium with illumination light emitted from a light source 132a in a light source apparatus 132, described below. The light guide 111a extends to a distal end portion of the insertion section 111 from a proximal end portion of the universal cord section 113 via the universal cord section 113, the operation section 112, and the insertion section 111 to guide the illumination light into the predetermined region 143 from the light source 132a. An illumination lens may be provided at a distal end of the light guide 111a.

The imaging optical system 111b includes one or more lenses, and collects light from the predetermined region 143, to form an optical image of the predetermined region 143 in the image sensor 111c. The imaging optical system 111b may further have a movement structure for moving at least some of the one or more lenses included in the imaging optical system 111b in an optical axis direction. The imaging optical system 111b may realize an optical zoom function for changing a projection magnification of the optical image and a focus function for moving a focus position by the movement structure. The imaging optical system 111b may include one or more variable focus lenses capable of varying a focal length by changing a lens shape, and may realize an optical zoom function and a focus function by the one or more variable focus lenses included in the imaging optical system 111b. The imaging optical system 111b may realize the functions by combining the movement structure and the variable focus lenses.

The image sensor 111c is an example of a light receiving section that receives the optical image formed by the imaging optical system 111b, and is a two-dimensional image sensor such as a CCD (charge-coupled device) image sensor or a CMOS (complementary metal oxide semiconductor) image sensor, for example. The image sensor 111c receives light from the predetermined region 143 on a light receiving surface via the imaging optical system 111b, and converts the received light into an electric signal to generate an imaging signal of the predetermined region 143.

More specifically, in the image sensor 111c, a plurality of pixels each including a photodiode and a capacitor are two-dimensionally arranged, for example. Each of the pixels may further include a color filter. The color filters respectively arranged in the plurality of pixels are arranged in a Bayer array, for example. Instead of the color filters, a plurality of photodiodes may be laminated in a thickness direction in each of the pixels. The image sensor 111c reads out an electric signal generated by photoelectric conversion from the plurality of pixels, and outputs the electric signal to an image processing apparatus 131 via a signal line 111d as an imaging signal.

The recovery apparatus 120 includes a recovery nozzle 121, a recovery container 122, and a suction device 123, as illustrated in FIG. 2. The recovery nozzle 121 is an example of a second insertion section to be inserted into the culture medium in the container 140, and has its one distal end and other distal end respectively inserted into the container 140 and the recovery container 122. A distal end on the container 140 side of the recovery nozzle 121 is previously positioned relative to the predetermined region 143. Although the recovery nozzle 121 is arranged such that a recovery region overlaps the predetermined region 143 in this example, the recovery region may be positioned downstream of the predetermined region 143 in a flow of the culture medium, for example.

The recovery nozzle 121 is provided with a channel 121a, as illustrated in FIG. 3. The channel 121a is a conduit through which the sucked cell, together with the culture medium, passes. The recovery apparatus 120 recovers the cell in the medium from the container 140 via the channel 121a. A plurality of channels 121a may be formed in the recovery nozzle 121. The inner diameter of the channel 121a is desirably larger than the diameter of the cell to be recovered.

The recovery container 122 is a container that stores the cells recovered from the container 140. The recovery container 122 may be provided with a filter that further distinguishes the sorted cells depending on the size and other characteristics of each of the cells. That is, the cell recovery apparatus 100 may separate the cell to be recovered from other objects and recover the cell by a physical structure of a filter provided in the recovery container 122 in addition to object detection, described below, to be performed by the control apparatus 130. As the filter, a mesh filter, a hollow fiber filter, or the like that can distinguish the cells and the other objects depending on the size and the other characteristics may be used.

The suction device 123 is an example of a suction section that generates a suction force. The suction device 123 may be able to generate a sufficient suction force to suck the cell to be recovered, and may be a suction pump that sucks air in the recovery container 122 to apply a negative pressure to the recovery container 122, for example. The suction device 123 may be a liquid feeding pump such as a peristaltic pump, and may act on the recovery nozzle 121 between the recovery container 122 and the container 140 with both ends of the recovery nozzle 121 immersed in the culture medium.

The suction device 123 is controlled by the control apparatus 130. The suction device 123 may start an operation by receiving a signal from the control apparatus 130 and further stop the operation by receiving the signal from the control apparatus 130, for example. When the suction device 123 operates, a suction force is generated at a distal end of the recovery nozzle 121 via the channel 121a. The recovery apparatus 120 sucks the cell in the medium from the container 140 with the suction force generated by the suction device 123, and outputs the sucked cell to the recovery container 122 via the channel 121a.

A return flow passage may be formed between the container 140 and the recovery container 122, although not illustrated, and the culture medium, together with the cells, flowing into the recovery container 122 may be returned to the container 140. Thus, the culture medium can be prevented from being uselessly consumed. A return flow structure is not particularly limited. For example, when the recovery container 122 may have a two-layer structure using a filter as a boundary, and the suction device 123, described below, acts on each of layers, a flow to the layer of the recovery container 122 in front of the filter from the container 140 and a flow from the layer of the recovery container 122 behind the filter to the container 140 may be formed.

The control apparatus 130 includes the image processing apparatus 131 that processes an image acquired using the observation apparatus 110, the light source apparatus 132 that supplies illumination light to the observation apparatus 110, and a display apparatus 133 that displays an image, as illustrated in FIG. 2.

The image processing apparatus 131 includes a signal processing unit 131a and an image analysis unit 131b, as illustrated in FIG. 3. The signal processing unit 131a performs predetermined processing for the imaging signal received from the observation apparatus 110, to generate an image signal. The predetermined processing may include noise reduction processing, analog-to-digital conversion processing, OB subtraction processing, WB correction processing, demosaicing processing, and color matrix processing, for example.

The image analysis unit 131b analyzes an image of the predetermined region 143 based on the image signal received from the signal processing unit 131a. The image analysis to be performed by the image analysis unit 131b may be object detection for detecting the cell to be recovered from inside the image, for example, and the object detection may be performed using a learned model obtained by previously learning the cell to be recovered using deep layer learning. When the image analysis unit 131b detects the cell to be recovered, the image processing apparatus 131 outputs a control signal to the recovery apparatus 120 to control the recovery apparatus 120 so that the recovery apparatus 120 recovers the cell. The image processing apparatus 131, the signal processing unit 131a, and the image analysis unit 131b may be each composed of a processor in the control apparatus 130, for example, although not particularly limited. If a communication delay occurring on a network falls within an acceptable degree, the image processing apparatus 131, the signal processing unit 131a, and the image analysis unit 131b may be composed of a user terminal 12 and other servers to be connected to the control apparatus 130 via a network such as the Internet.

The light source apparatus 132 includes the light source 132a and a light source driving unit 132b, as illustrated in FIG. 3. The light source 132a is a light source that emits illumination light to be supplied to the observation apparatus 110. The light source 132a is an LED light source, for example, and is not limited to the LED light source, but may be a lamp light source such as a xenon lamp or a halogen lamp or may be a laser light source. The light source 132a may include a plurality of LED light sources that respectively emit illumination light beams having different colors.

The light source driving unit 132b is a driver that drives the light source 132a, for example, an LED driver. The light source driving unit 132b drives the light source 132a according to setting of the light source apparatus 132. An instruction to set the light source apparatus 132 may be issued by an instruction value (e.g., a current value or a voltage value) from the control apparatus 130, or the instruction may be issued by an operation for a switch provided in the light source apparatus 132, for example.

The display apparatus 133 displays an image acquired using the observation apparatus 110 and the control apparatus 130. The display apparatus 133 include any display including a liquid crystal display or an organic EL display, for example. An image may be displayed on not the display apparatus 133 but the user terminal 12.

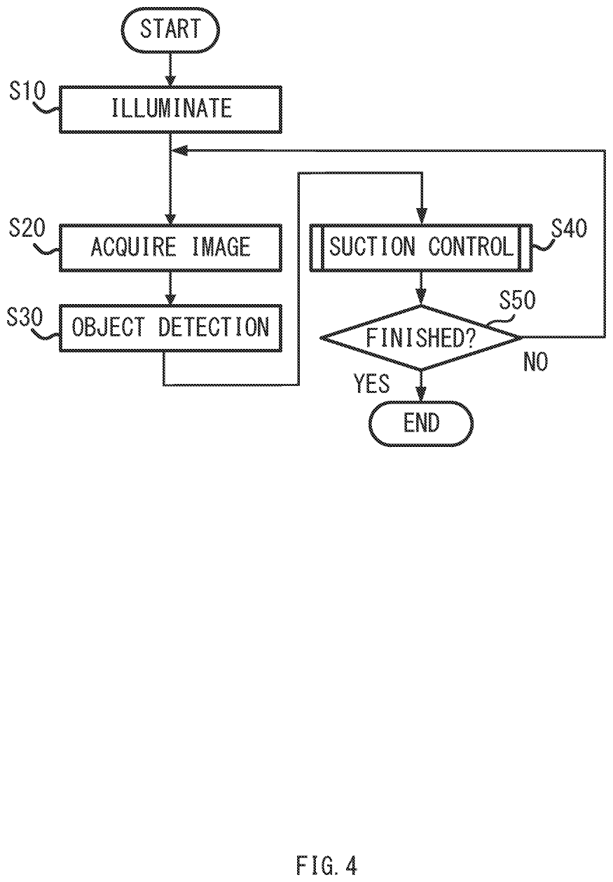

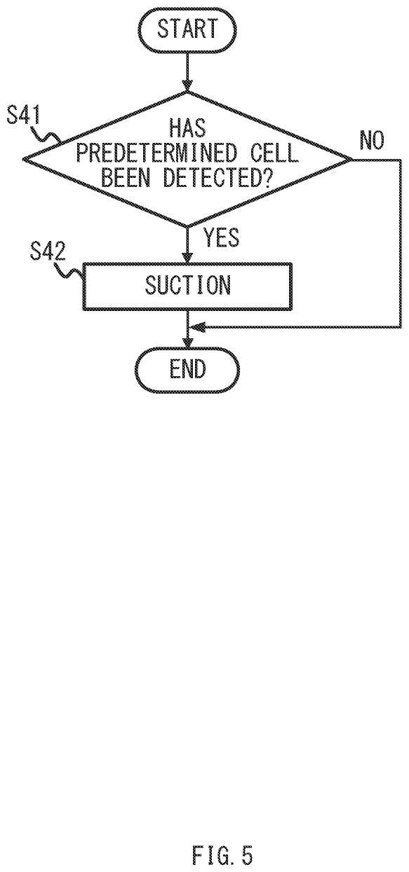

FIG. 4 is a flowchart illustrating an example of cell recovery processing to be performed by the cell recovery apparatus 100. FIG. 5 is a flowchart illustrating an example of suction control processing to be performed by the cell recovery apparatus 100. A cell recovery method to be performed using the cell recovery apparatus 100 will be described below with reference to FIG. 4 and FIG. 5.

The cell recovery processing illustrated in FIG. 4 is started according to a start instruction from the user and a set schedule, for example, when the processor in the control apparatus 130 executes a cell recovery program. At this time, the insertion section 111 in the observation apparatus 110 and the recovery nozzle 121 in the recovery apparatus 120 are previously inserted into the container 140 before the cell recovery processing is started. More specifically, the insertion section 111 and the recovery nozzle 121 are positioned such that the recovery region overlaps the predetermined region 143 (an observation region), for example.

The cell recovery processing may be performed while continuing to stir the culture medium or after stopping stirring the culture medium. When an approximately height at which the cell to be recovered is suspended while the culture medium is agitated can be expected, the height of the predetermined region 143 may be made to match the height to be expected, and the cell recovery apparatus 100 may recover the cell while stirring the culture medium. For example, the height of the predetermined region 143 may be made to match the vicinity of a bottom surface of the container 140, and the cell recovery apparatus 100 may recover the cell that has settled to the vicinity of the bottom surface by stopping stirring the culture medium.

When the cell recovery processing is started, the cell recovery apparatus 100 first illuminates the predetermined region 143 (step S10). In step S10, the light source apparatus 132 emits illumination light from the light source 132a, and the observation apparatus 110 irradiates the predetermined region 143 with illumination light guided to the light guide 111a.

Then, the cell recovery apparatus 100 acquires an image of the predetermined region 143 (step S20). In step S20, the observation apparatus 110 images the predetermined region 143, and the image processing apparatus 131 generates the image of the predetermined region 143 based on an imaging signal received from the observation apparatus 110. That is, the control apparatus 130 acquires the image of the predetermined region 143 using the observation apparatus 110.

When the image of the predetermined region 143 is acquired, the cell recovery apparatus 100 performs object detection for the acquired image (step S30). In step S30, the image processing apparatus 131 performs the object detection using a learned model obtained by learning at least the cell to be recovered.

Then, the cell recovery apparatus 100 performs suction control processing illustrated in FIG. 5 (step S40). In step S40, the control apparatus 130 determines whether or not the predetermined cell, i.e., the cell to be recovered has been detected by the object detection in step S30 (step S41), and controls the recovery apparatus 120 depending on a determination result. Specifically, when the cell to be recovered has been detected, the control apparatus 130 instructs the recovery apparatus 120 to perform suction, and causes the recovery apparatus 120 to recover the detected cell (step S42). On the other hand, when the cell to be recovered has not been detected, the control apparatus 130 does not instruct the recovery apparatus 120 to perform suction. In other words, the control apparatus 130 causes the recovery apparatus 120 to perform a recovery operation when it detects the cell to be recovered by the object detection, and does not cause the recovery apparatus 120 to perform the recovery operation when it does not detect the cell to be recovered by the object detection.

When the suction control processing ends, the cell recovery apparatus 100 determines whether or not the cell recovery processing ends (step S50). In step S50, the cell recovery apparatus 100 may determine whether or not cell recovery processing ends according to a predetermined rule (e.g., an elapse of a predetermined time period from the start of the cell recovery processing and an end instruction from the user). The cell recovery apparatus 100 repeatedly performs the processing in step S20 to step S40 until it is determined in step S50 that the cell recovery processing ends, and finishes the cell recovery processing when it is determined in step S50 that the cell recovery processing ends.

When the cell recovery apparatus 100 is used, as described above, the user can directly recover the cell to be recovered from a culture environment only by instructing the cell recovery apparatus 100 to recover the cell. In the case, when the cells suspended in the culture medium are sucked, the cells can be recovered without being excessively stressed. Therefore, the cell recovery apparatus 100 makes it possible to sort the cells efficiently and while suppressing a damage to the cells without imposing a burden of recovery work on the user.

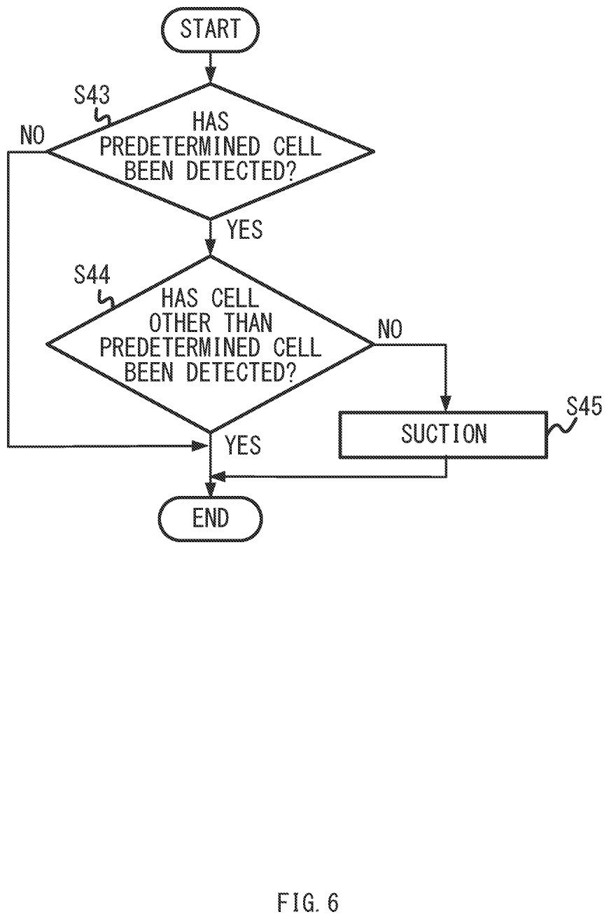

FIG. 6 is a flowchart illustrating another example of suction control processing to be performed by the cell recovery apparatus 100. The cell recovery apparatus 100 may perform the suction control processing illustrated in FIG. 6 instead of the suction control processing illustrated in FIG. 5. The suction control processing illustrated in FIG. 6 differs from the suction control processing illustrated in FIG. 5 in that the absence of the cells other than the cell to be recovered is also confirmed in addition to the presence of the cell to be recovered. In the control apparatus 130, object detection is performed using a learned model obtained by learning objects other than the cell to be recovered (e.g., the cells other than the cell to be recovered) in addition to the cell to be recovered.

When the suction control processing illustrated in FIG. 6 is started, the control apparatus 130 determines whether or not the predetermined cell, i.e., the cell to be recovered has been detected by the object detection in step S30 illustrated in FIG. 4 (step S43). If it is determined in step S43 that the cell to be recovered has been detected, the control apparatus 130 further determines whether or not the cells other than the cell to be recovered have been detected (step S44). If it is determined in step S44 that the cells other than the cell to be recovered have not been detected, the control apparatus 130 instructs the recovery apparatus 120 to perform suction, and causes the recovery apparatus 120 to recover the detected cell to be recovered (step S45). On the other hand, if the cell to be recovered has not been detected and the cells other than the cell to be recovered have been detected, the control apparatus 130 does not instruct the recovery apparatus 120 to perform suction. In other words, the control apparatus 130 causes the recovery apparatus 120 to perform a recovery operation when the cell to be recovered has been detected and the cells other than the cell to be recovered have not been detected by the object detection, does not cause the recovery apparatus 120 to perform a recovery operation when the cell to be recovered has not been detected by the object detection, and does not cause the recovery apparatus 120 to perform the recovery operation when the cells other than the cell to be recovered have been detected by the object detection.

Even if the cell recovery apparatus 100 performs the suction control processing illustrated in FIG. 6 instead of the suction control processing illustrated in FIG. 5, the user can directly recover the cell to be recovered from the culture environment only by instructing the cell recovery apparatus 100 to recover the cell. Particularly, in the suction control processing illustrated in FIG. 6, a recovery operation is performed after confirming not only the presence of the cell to be recovered but also the absence of the cells other than the cell to be recovered. Accordingly, the cell recovery apparatus 100 makes it possible to significantly reduce the possibility that the cells not to be recovered, together with the cell to be recovered, are recovered. Therefore, the cells can be sorted with high accuracy.

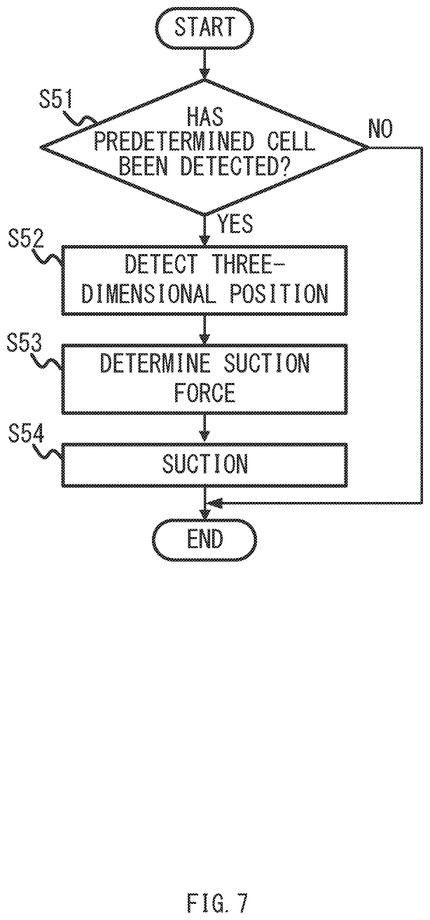

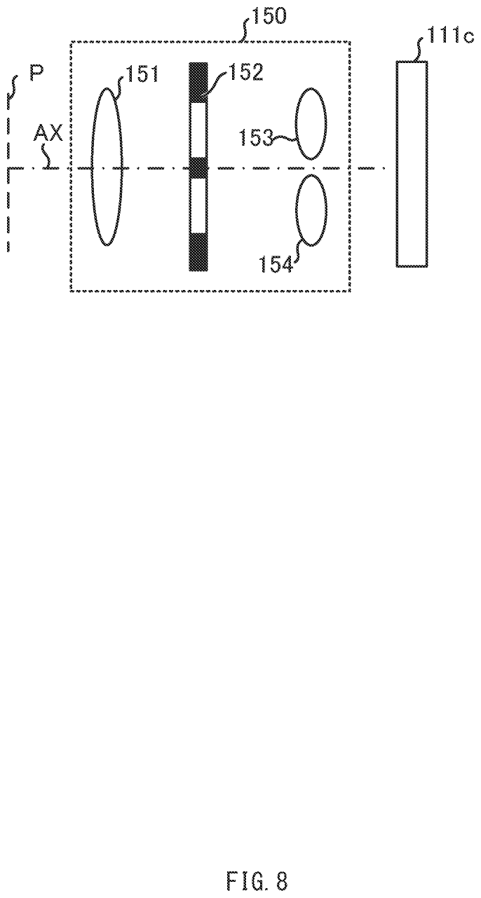

FIG. 7 is a flowchart illustrating still another example of suction control processing to be performed by the cell recovery apparatus 100. FIG. 8 is a diagram illustrating a stereo optical system 150. The cell recovery apparatus 100 may perform the suction control processing illustrated in FIG. 7 instead of the suction control processing illustrated in FIG. 5. The suction control processing illustrated in FIG. 7 differs from the suction control processing illustrated in FIG. 5 in that a suction force is controlled depending on a position of the detected cell to be recovered. Accordingly, the observation apparatus 110 may include the stereo optical system 150 illustrated in FIG. 8 in the imaging optical system 111b as means for detecting the position of the cell to be recovered, and the control apparatus 130 may acquire two images having a parallax using the observation apparatus 110 to stereographically measure the position of the cell to be recovered.

The stereo optical system 150 may include an objective lens 151, a diaphragm 152 having two apertures eccentric with respect to an optical axis AX, and two imaging lenses 153 that collect light beams that have respectively passed through the two apertures to form an optical image of an observation surface P on the image sensor 111c, for example, as illustrated in FIG. 8. The stereo optical system 150 illustrated in FIG. 8 is only an example of a stereo optical system forming optical images having a parallax. The observation apparatus 110 may include a stereo optical system having a different configuration from that of the stereo optical system 150.

The cell recovery apparatus 100 may be able to detect a position of an object detected by the object detection, and may detect the position of the object by means other than the stereo optical system. The cell recovery apparatus 100 may include a distance image sensor of a TOF (time of flight) type, for example, and may detect the position of the object based on a distance image.

When the suction control processing illustrated in FIG. 7 is started, the control apparatus 130 determines whether or not the predetermined cell, i.e., the cell to be recovered has been detected by the object detection in step S30 illustrated in FIG. 4 (step S51). When it is determined in step S51 that the cell to be recovered has been detected, the control apparatus 130 detects a three-dimensional position of the detected cell to be recovered (step S52). The control apparatus 130 detects the three-dimensional position of the cell to be recovered from the images having a parallax acquired using the stereo optical system, for example.

When the three-dimensional position of the cell to be recovered is detected, the control apparatus 130 determines a suction force for sucking the cell to be recovered (step S53). The control apparatus 130 may determine the suction force to a value that increases as a distance of the three-dimensional position of the cell to be recovered detected in step S52 from the distal end of the recovery nozzle 121 increases, for example. When a plurality of cells to be recovered have been detected, the suction force may be determined based on the three-dimensional position of the cell farthest away from the distal end of the recovery nozzle 121. When the suction force is determined, the control apparatus 130 controls the recovery apparatus 120 to suck the cell to be recovered with the suction force determined in step S53 (step S54). That is, the control apparatus 130 causes the recovery apparatus 120 to perform a recovery operation using the suction force corresponding to the three-dimensional position of the cell to be recovered.

Even when the cell recovery apparatus 100 performs the suction control processing illustrated in FIG. 7 instead of the suction control processing illustrated in FIG. 5, as described above, the cell recovery apparatus 100 can recover the cells suspended in the culture medium without excessively stressing the cells by sucking the cells. Particularly in the suction control processing illustrated in FIG. 7, the cell to be recovered can be recovered with a minimum necessary suction force depending on a three-dimensional position of the cell. Accordingly, a stress on the cell can be more suppressed than when the suction control processing illustrated in FIG. 5 is performed. The suction force can be avoided being not only excessive but also insufficient. Accordingly, cell recovery work can be efficiently performed by more reliably recovering the cell.

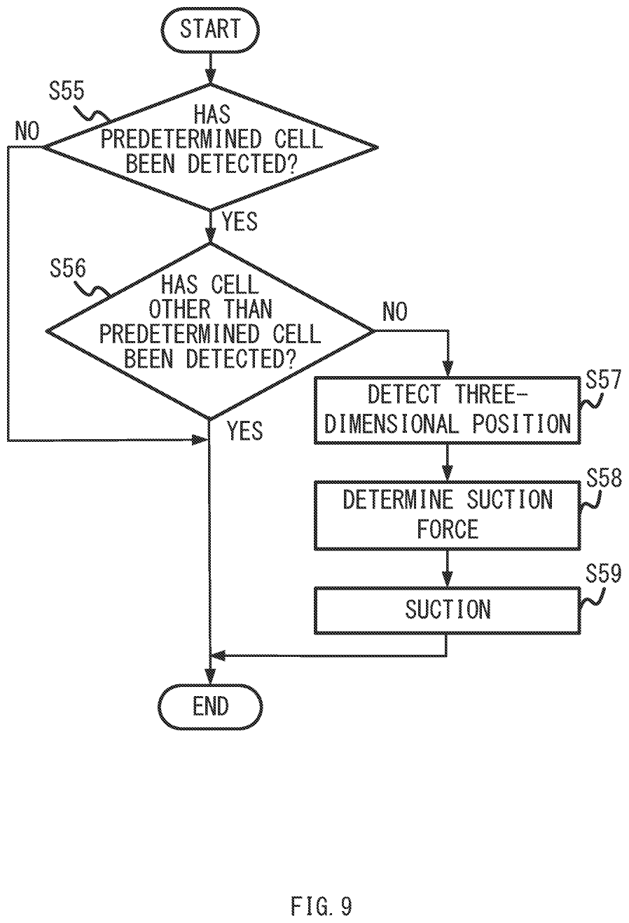

FIG. 9 is a flowchart illustrating still another example of suction control processing to be performed by the cell recovery apparatus 100. The cell recovery apparatus 100 may perform the suction control processing illustrated in FIG. 9 instead of the suction control processing illustrated in FIG. 5. The suction control processing illustrated in FIG. 9 corresponds to a combination of the suction control processing illustrated in FIG. 6 and the suction control processing illustrated in FIG. 7. That is, the suction control processing illustrated in FIG. 9 differs from the suction control processing illustrated in FIG. 5 in that the absence of the cells other than the cell to be recovered is also confirmed in addition to the presence of the cell to be recovered and in that a suction force is controlled depending on a three-dimensional position of the detected cell to be recovered.

When the suction control processing illustrated in FIG. 9 is started, the control apparatus 130 determines whether or not the predetermined cell, i.e., the cell to be recovered has been detected by object detection (step S55). If it is determined that the cell to be recovered has been detected, the control apparatus 130 further determines whether or not the cells other than the cell to be recovered have been detected (step S56). The processing in step S55 and step S56 is similar to the processing in step S43 and step S44 illustrated in FIG. 6.

If it is determined in step S56 that the cells other than the cell to be recovered have not been detected, the control apparatus 130 detects a three-dimensional position of the detected cell to be recovered (step S57), determines a suction force for sucking the cell to be recovered (step S58), and controls the recovery apparatus 120 to suck the cell to be recovered with the determined suction force (step S59). The processing in step S57 to step S59 is similar to the processing in step S52 to step S54 illustrated in FIG. 7.

When the cell recovery apparatus 100 performs the suction control processing illustrated in FIG. 9 instead of the suction control processing illustrated in FIG. 5, as described above, the possibility that the cells other than the cell to be recovered, together with the cell to be recovered, are recovered can be significantly reduced while more suppressing a stress on the cells than when the suction control processing illustrated in FIG. 5 is performed.

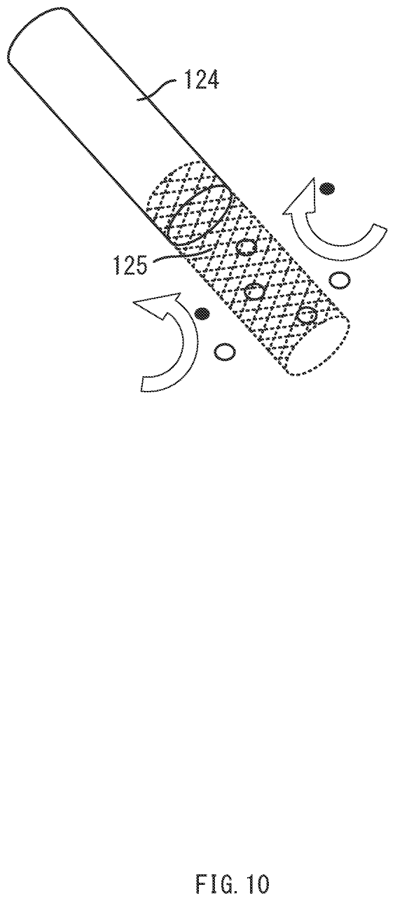

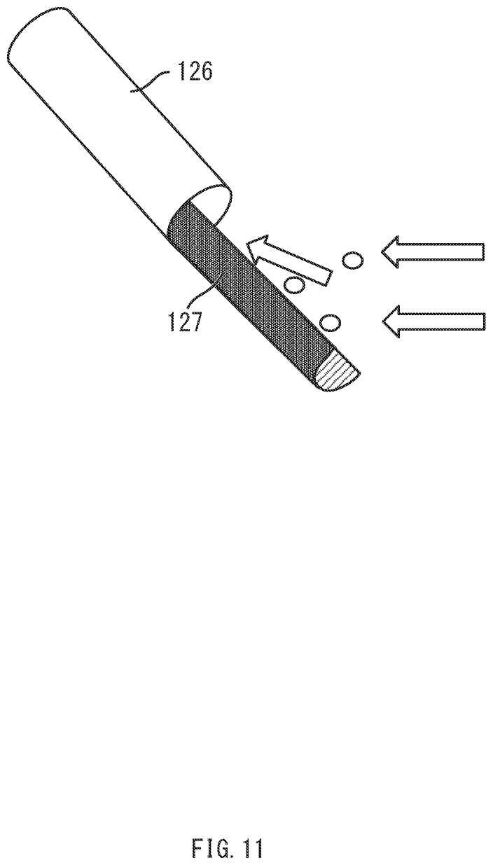

FIG. 10 is a diagram illustrating a configuration of a recovery nozzle 124. FIG. 11 is a diagram illustrating a configuration of a recovery nozzle 126. The recovery apparatus 120 may include a partition section that partitions, to support the recovery of the cell to be recovered that has been detected by object detection, a region in the medium 6 where the cells are to be recovered by the recovery apparatus 120. The recovery nozzle 124 illustrated in FIG. 10 and the recovery nozzle 126 illustrated in FIG. 11 are each an example of a recovery nozzle including the above-described partition section, and the recovery apparatus 120 may include the recovery nozzle 124 or the recovery nozzle 126 instead of the recovery nozzle 121.

The recovery nozzle 124 illustrated in FIG. 10 has a cylindrical net 125 provided at its distal end. The net 125 is an example of the partition section that partitions a region in the medium 6 where the cells are to be recovered, and an object outside the net 125 can be avoided being sucked in the recovery nozzle 124 when the net 125 is provided. That is, a recovery target can be controlled by restricting a recovery range. Accordingly, the possibility that an unnecessary object is recovered, together with the cell to be recovered, can be reduced.

The recovery nozzle 126 illustrated in FIG. 11 has a control plate 127 having a shape obtained by cutting a cylinder along an axis provided at its distal end. The control plate 127 is an example of a partition section that partitions a region in the medium 6 where the cells are to be recovered. When the control plate 127 is provided, the cell that moves in a direction nearer to the channel 121a with a suction force can be guided into the channel 121a. Accordingly, a recovery loss of the cell to be recovered that has been detected by the object detection can be reduced. The control plate 127 is desirably arranged downstream of the predetermined region 143 in a flow of the culture medium. As a result, the cell on which the flow of the culture medium acts in addition to the suction force can be guided into the channel 121a along the control plate 127.

### Second Embodiment

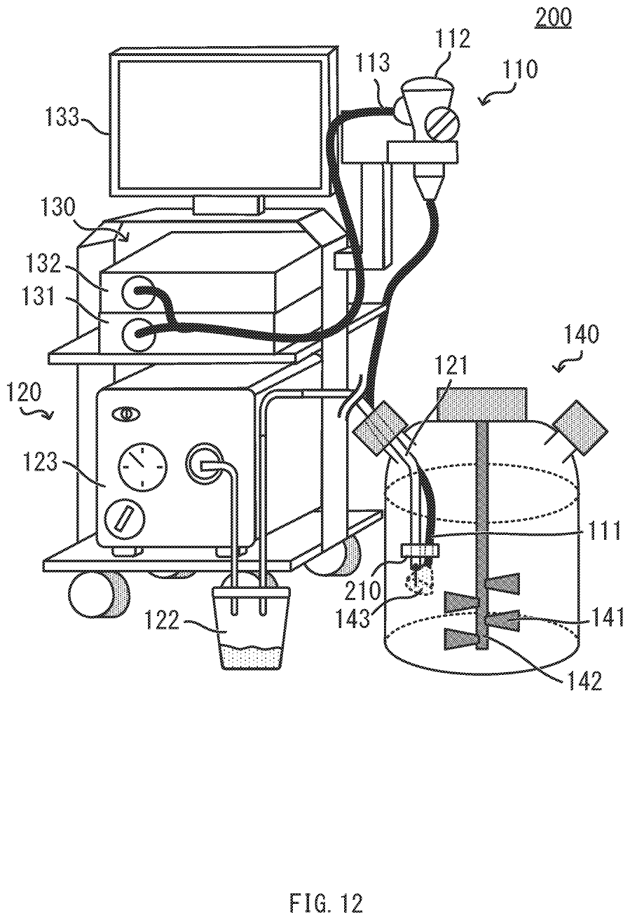

FIG. 12 is a diagram illustrating a configuration of a cell recovery apparatus 200 according to the present embodiment. The cell recovery apparatus 200 is an apparatus that automatically sorts cells cultured in suspension culture. The cell recovery apparatus 200 is similar to the cell recovery apparatus 100 in that it includes an observation apparatus 110, a recovery apparatus 120, and a control apparatus 130.

The cell recovery apparatus 200 differs from the cell recovery apparatus 100 in that it includes a bundle member 210 that bundles an insertion section 111 and a recovery nozzle 121. In the cell recovery apparatus 200, the recovery nozzle 121 is positioned in a predetermined relationship with the insertion section 111 using the bundle member 210.

The cell recovery apparatus 200 according to the present embodiment also makes it possible to sort the cells efficiently and while suppressing a damage to the cells without imposing a burden of recovery work on a user, like the cell recovery apparatus 100. The cell recovery apparatus 200 makes it possible to support positioning work to be performed by the user by using the bundle member 210.

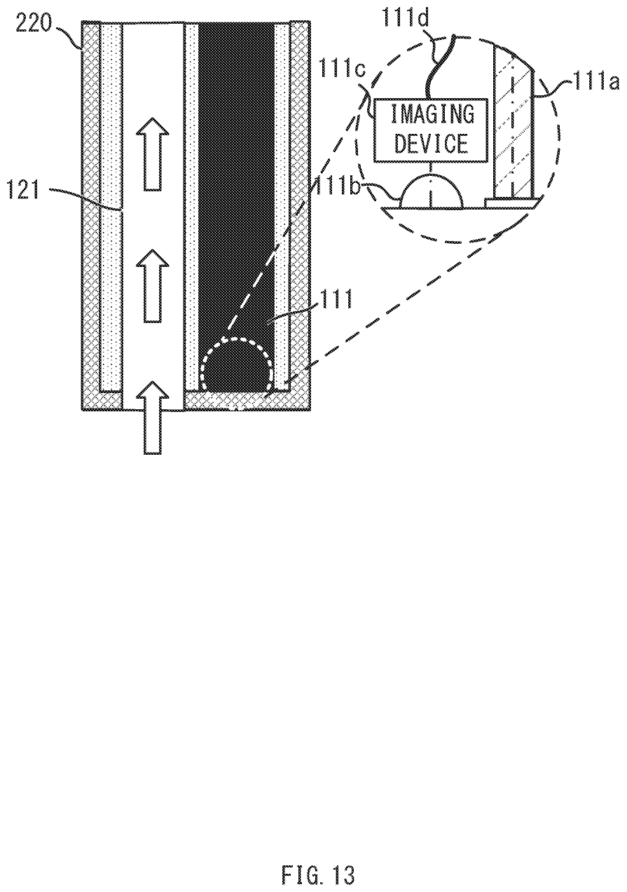

FIG. 13 is a diagram for describing a modification of the cell recovery apparatus 200. Although an example in which the cell recovery apparatus 200 supports the positioning work using the bundle member 210 is illustrated in FIG. 12, the cell recovery apparatus 200 may include a housing 220 illustrated in FIG. 13 instead of the bundle member 210. The housing 220 accommodates the insertion section 111 and the recovery nozzle 121, and the insertion section 111 and the recovery nozzle 121 are maintained in a predetermined positional relationship in the housing 220.

A distal end of the insertion section 111 is inserted into an opening formed in the housing 220. Preferably, a culture medium can be avoided entering the housing 220 from the opening formed in the housing 220 by arranging an O ring, for example, in a gap between the distal end of the insertion section 111 and the housing 220.

The cell recovery apparatus 200 can also support positioning work to be performed by the user by using the housing 220 instead of the bundle member 210. In the cell recovery apparatus 200, when a housing of a disposable type that has already been sterilized is used for the housing 220, a burden of maintenance work such as cleaning and disinfection of the cell recovery apparatus 200 can be reduced. Specifically, the insertion section 111 does not contact the culture medium by using the housing 220. Accordingly, cleaning and disinfection of the insertion section 111 are omitted or replaced with simple cleaning and simple disinfection, for example.

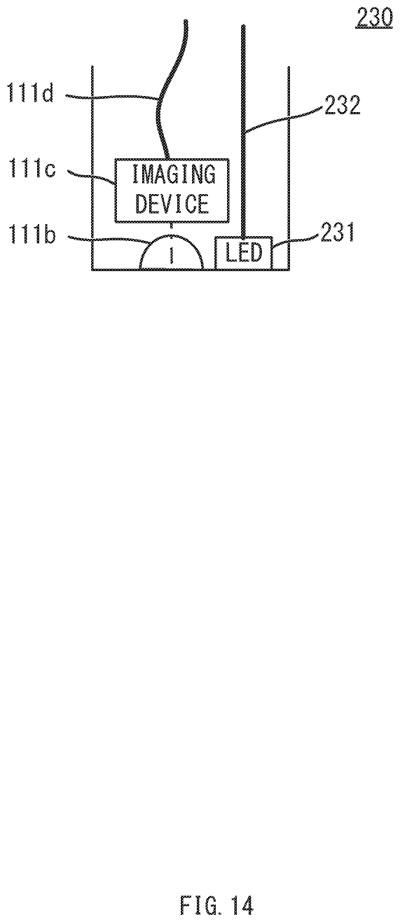

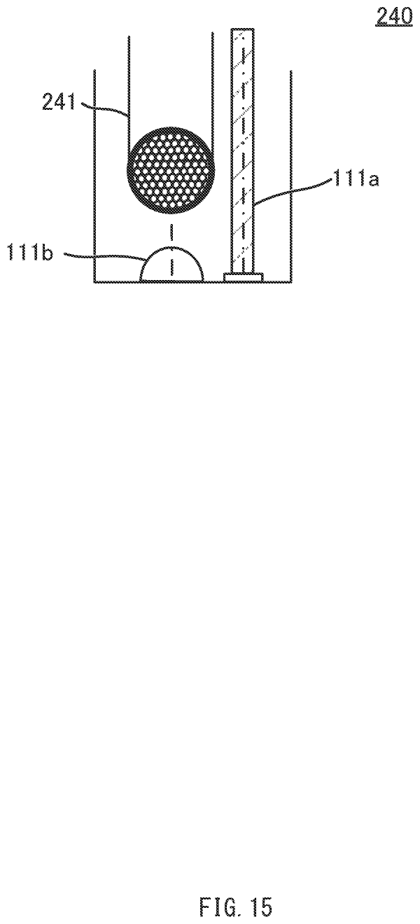

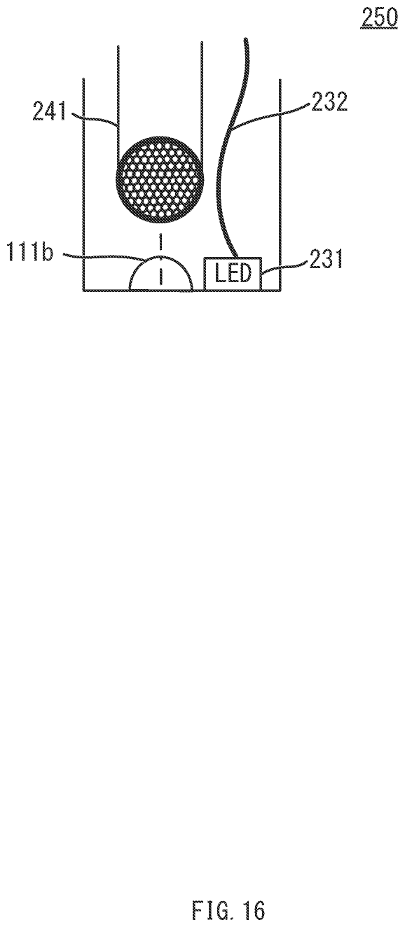

FIG. 14 is a diagram illustrating a configuration of an insertion section 230. FIG. 15 is a diagram illustrating a configuration of an insertion section 240. FIG. 16 is a diagram illustrating a configuration of an insertion section 250. Modifications of a configuration of an insertion section will be respectively described below with reference to the FIG. 14 to FIG. 16.

The insertion section 230 illustrated in FIG. 14 differs from the insertion section 111 in that it includes an LED 231 and a control line 232 instead of a light guide 111a. The control line 232 is connected to the control apparatus 130, for example. The LED 231 is an example of an illumination section, and is controlled to emit light by the control apparatus 130.

The insertion section 240 illustrated in FIG. 15 differs from the insertion section 111 in that it includes a bundle optical fiber 241 instead of an image sensor 111c. The bundle optical fiber 241 is an example of a light receiving section that receives an optical image formed by an imaging optical system 111b, and relays an optical image of a predetermined region 143 to an image sensor not illustrated provided in the observation apparatus 110.

The insertion section 250 illustrated in FIG. 16 differs from the insertion section 111 in that it includes an LED 231 and a control line 232 instead of the light guide 111a and in that it includes a bundle optical fiber 241 instead of the image sensor 111c.

The observation apparatus 110 included in each of the cell recovery apparatus 100 according to the first embodiment and the cell recovery apparatus 200 according to the second embodiment may include any of the insertion sections illustrated in FIG. 14 to FIG. 16 instead of the insertion section 111.

### Third Embodiment

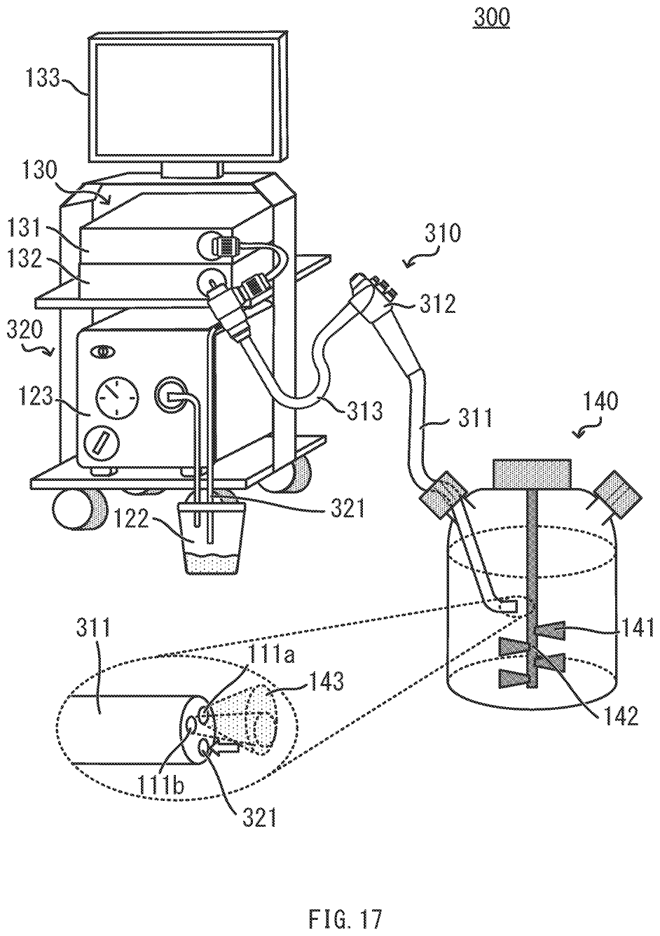

FIG. 17 is a diagram illustrating a configuration of a cell recovery apparatus 300 according to the present embodiment. The cell recovery apparatus 300 is an apparatus that automatically sorts cells cultured in suspension culture. The cell recovery apparatus 300 is similar to the cell recovery apparatus 100 in that it includes a control apparatus 130. The cell recovery apparatus 300 differs from the cell recovery apparatus 100 in that it includes an observation apparatus 310 instead of the observation apparatus 110 and in that it includes a recovery apparatus 320 instead of the recovery apparatus 120.

The observation apparatus 310 has a similar structure to that of the observation apparatus 110, and includes an insertion section 311, an operation section 312, and a universal cord section 313. The recovery apparatus 320 includes a recovery nozzle 321, a recovery container 122, and a suction device 123.

The observation apparatus 310 differs from the observation apparatus 110 in that a channel that penetrates at least the insertion section 311 is formed. The recovery apparatus 320 differs from the recovery apparatus 120 in that the channel of the observation apparatus 310 constitutes a channel of the recovery nozzle 321.

That is, the cell recovery apparatus 300 differs from the cell recovery apparatus 100 in that the recovery apparatus 320 has the channel provided in the insertion section 311 in the observation apparatus 310 and the recovery nozzle 321 is inserted into a container 140 via the observation apparatus 310. As a result, in the cell recovery apparatus 300, a position of the recovery nozzle 321 is always maintained in a predetermined relationship with a predetermined region 143, as illustrated in FIG. 17. Accordingly, the recovery apparatus 320 is positioned relative to the predetermined region 143 without positioning work by a user.

The observation apparatus 310 may be an existing medical or industrial endoscope, and an existing channel such as a forceps channel and an air feeding/water feeding channel of the endoscope may be used as the channel of the recovery nozzle 321.

The cell recovery apparatus 300 according to the present embodiment also makes it possible to sort the cells efficiently and while suppressing a damage to the cells without imposing a burden of recovery work on the user, like the cell recovery apparatus 100. The cell recovery apparatus 300 makes it possible for the user to use the cell recovery apparatus 300 without performing positioning work of the recovery apparatus 320 relative to the predetermined region 143.

### Fourth Embodiment

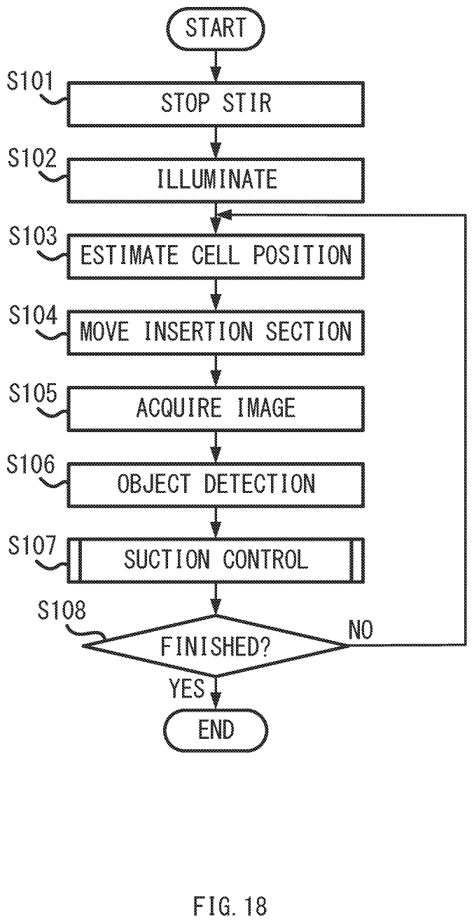

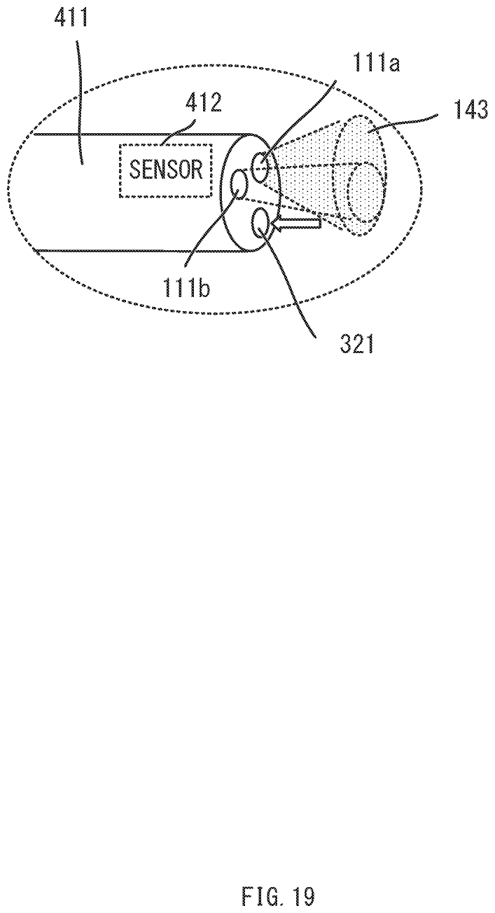

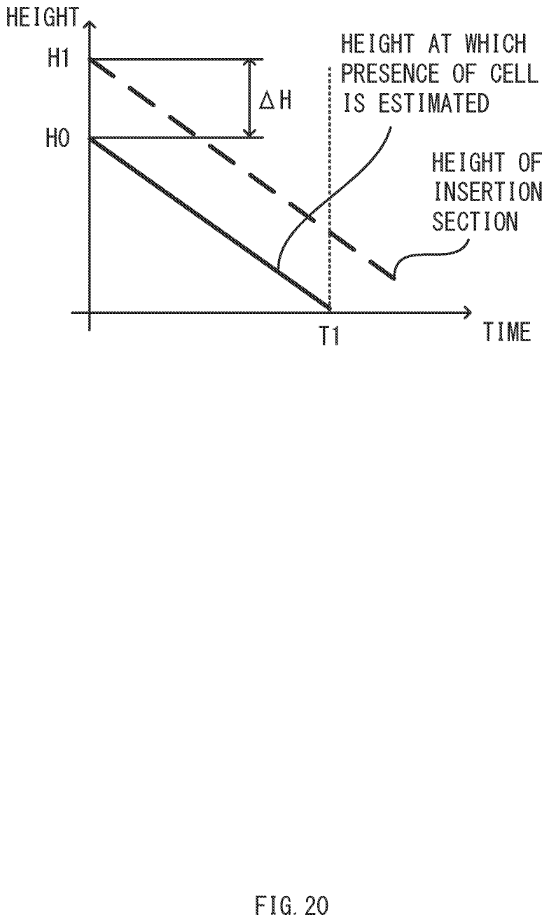

FIG. 18 is a flowchart illustrating an example of cell recovery processing to be performed by a cell recovery apparatus according to the present embodiment. FIG. 19 is a diagram illustrating a configuration of an insertion section 411. FIG. 20 is a diagram for describing an example of height control of the insertion section 411. The cell recovery apparatus according to the present embodiment is an apparatus that automatically sorts cells cultured in suspension culture. The cell recovery apparatus according to the present embodiment differs from the respective cell recovery apparatuses according to the above-described embodiments in that it estimates a region where the cells exist in a container 140 and an insertion section moves in a medium depending on an estimation result.

An apparatus configuration of the cell recovery apparatus according to the present embodiment differs from the cell recovery apparatus 300 in that it includes the insertion section 411 instead of the insertion section 311, as illustrated in FIG. 19. Other points are similar to those in the cell recovery apparatus 300. The insertion section 411 differs from the insertion section 311 in that the insertion section 411 is provided with a sensor 412 for detecting a distal end position of the insertion section 411 and in that a position of the insertion section 411 is controlled by a control apparatus 130. The sensor 412 is a three-dimensional position sensor, for example. A method of measuring a three-dimensional position is not particularly limited. The sensor 412 may include a three-axis acceleration sensor and a three-axis angular velocity sensor, or may include a magnetic sensor to be used in combination with a magnetic field generation device not illustrated.

Information detected by the sensor 412 may be outputted to the control apparatus 130 and used for position control of the insertion section 411, for example. The information detected by the sensor 412 may be used for position control of the insertion section 411 in an observation apparatus 310, for example.

The cell recovery processing illustrated in FIG. 18 is started according to a start instruction from a user or a set schedule, for example, when a processor in the control apparatus 130 executes a cell recovery program. At this time, the insertion section 411 in the observation apparatus is previously inserted into the container 140 before the cell recovery processing is started.

When the cell recovery processing is started, the cell recovery apparatus first stops stirring a culture medium (step S101). For example, the control apparatus 130 connected to the container 140 stops a motor of the container 140, to stop stirring the culture medium. The user may manually stop the stir.

Then, the cell recovery apparatus illuminates a predetermined region 143 (step S102). In step S102, a light source apparatus 132 emits illumination light from a light source 132a, and the observation apparatus irradiates the predetermined region 143 with the illumination light.

Then, the cell recovery apparatus estimates an existence position of the cell to be recovered in the container 140 (step S103), and moves the insertion section 411 depending on the estimated position (step S104). In step S103, the control apparatus 130 estimates the position of the cell to be recovered (referred to by an estimated cell position) based on a size and a mass to be assumed of the cell to be recovered and a time period elapsed since the stir was stopped, for example. In step S104, the control apparatus 130 determines a position after movement of the insertion section 411 based on the estimated cell position, and moves the insertion section 411 to the determined position.

The position after the movement of the insertion section 411 may differ from the estimated cell position. The position of the insertion section 411 may be a position suitable for recovering the cell existing in the estimated cell position, or may be a position spaced a predetermined distance apart from the estimated cell position in consideration of an orientation of the insertion section 411. As illustrated in FIG. 20, for example, the insertion section 411 may move to a position that is higher by a distance ΔH than the estimated cell position. When it is assumed that the cell has reached a bottom surface of the container 140, it is expected that a larger suction force is required than when the cell is suspended, for example, adhered. Accordingly, the insertion section 411 may recover the cell by coming closer thereto than the distance ΔH, as illustrated in FIG. 20.

Then, the cell recovery apparatus acquires an image of the predetermined region 143 (step S105), performs object detection for the acquired image (step S106), and performs suction control processing (step S107). The processing in step S105 to step S107 is similar to the processing in step S20 to step S40 illustrated in FIG. 4.

When the suction control processing ends, the cell recovery apparatus determines whether or not the cell recovery processing ends (step S108). The cell recovery apparatus repeatedly performs the processing in step S103 to step S107 until it is determined in step S108 the cell recovery processing ends, and finishes the cell recovery processing when it is determined in step S108 that the cell recovery processing ends.

The cell recovery apparatus according to the present embodiment also makes it possible to sort the cells efficiently and while suppressing a damage to the cells without imposing a burden of recovery work on the user, like the cell recovery apparatuses according to the above-described embodiments. Further, the cell recovery apparatus according to the present embodiment makes it possible to efficiently recover the cell to be recovered because the insertion section 411 appropriately moves to a position where the cell to be recovered is estimated to exist.

Although an example in which the cell is recovered with the stir stopped is illustrated in the present embodiment, cell recovery processing illustrated in FIG. 18 may be performed, when a position of the cell that is being stirred is estimated, during the stir.

### Fifth Embodiment

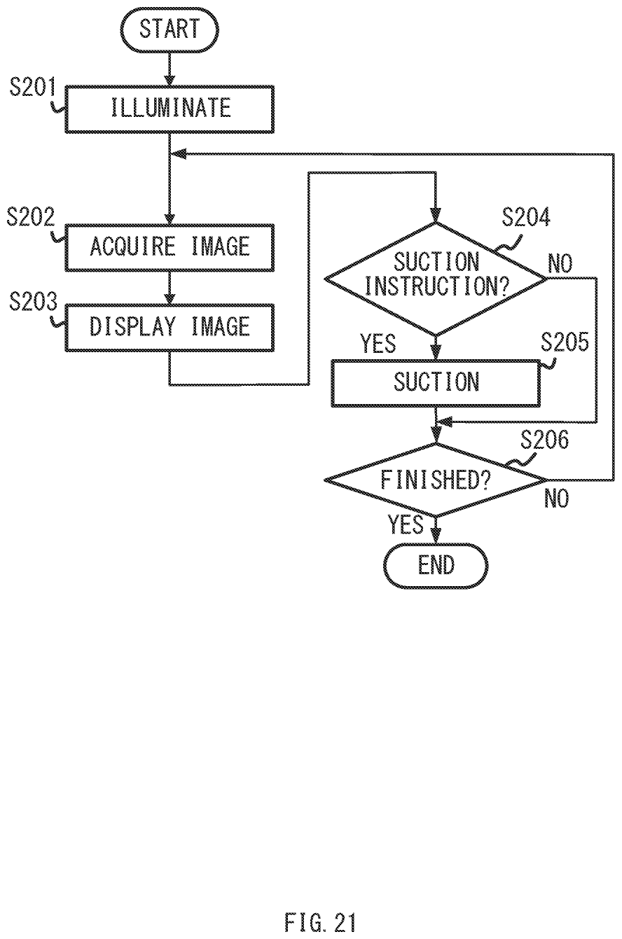

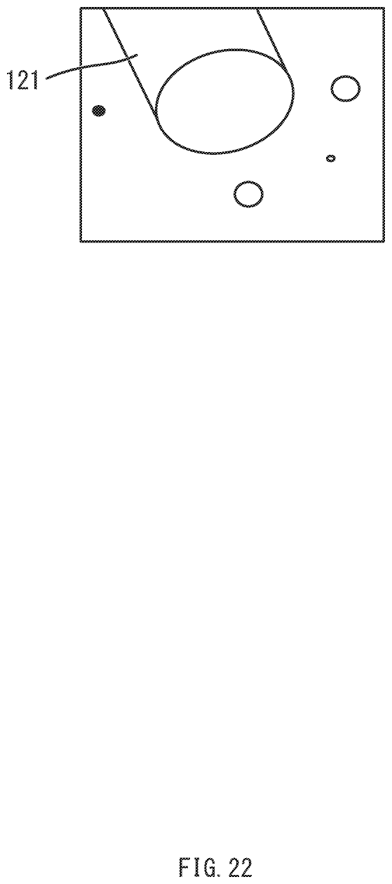

FIG. 21 is a flowchart illustrating an example of cell recovery processing to be performed by a cell recovery apparatus according to the present embodiment. FIG. 22 is a diagram illustrating an example of an image to be displayed on a display apparatus 133. The cell recovery apparatus according to the present embodiment is an apparatus that automatically sorts cells cultured in suspension culture.

An apparatus configuration of the cell recovery apparatus according to the present embodiment is similar to the cell recovery apparatus 100 illustrated in FIG. 2 except that a control apparatus 130 controls a recovery apparatus 120 in response to an instruction from a user. That is, the cell recovery apparatus according to the present embodiment differs from the cell recovery apparatus 100 in that the recovery apparatus 120 recovers the cell according to a manual operation to be performed by the user while seeing an image of a predetermined region 143.

The cell recovery processing illustrated in FIG. 21 is started in response to a start instruction from the user, for example, when a processor in the control apparatus 130 executes a cell recovery program. When the cell recovery processing is started, the cell recovery apparatus first illuminates the predetermined region 143 (step S201), and acquires an image of the predetermined region 143 (step S202). The processing in step S201 and step S202 is similar to the processing in step S10 and step S20 illustrated in FIG. 4.

When the image of the predetermined region 143 is acquired, the cell recovery apparatus displays the acquired image (step S203). The control apparatus 130 causes the display apparatus 133 to display an image illustrated in FIG. 22, for example. The user sees the image displayed on the display apparatus 133, to determine whether or not the cell to be recovered exists in a recovery region.

Then, the cell recovery apparatus determines the presence or absence of a suction instruction (step S204). The user confirms the presence of the cell to be recovered by seeing the image, and presses a suction button provided in an operation section 112, for example, the control apparatus 130 detects the suction instruction (YES in step S204), and performs suction control processing (step S205). The processing in step S205 is similar to the processing in step S40 illustrated in FIG. 4.

When the suction control processing ends, the cell recovery apparatus determines whether or not the cell recovery processing ends (step S206). The cell recovery apparatus repeatedly performs the processing in step S202 to step S205 until it is determined in step S206 that the cell recovery processing ends, and finishes the cell recovery processing when it is determined in step S206 that the cell recovery processing ends.

The cell recovery apparatus according to the present embodiment makes it possible to recover the cells suspended in a culture medium without excessively stressing the cells by sucking the cells. The user can sort the cells by performing a recovery operation while confirming the presence of the cell to be recovered by the image. Therefore, the cell recovery apparatus according to the above-described embodiment also makes it possible to sort the cells while suppressing a damage to the cells.

### Sixth Embodiment

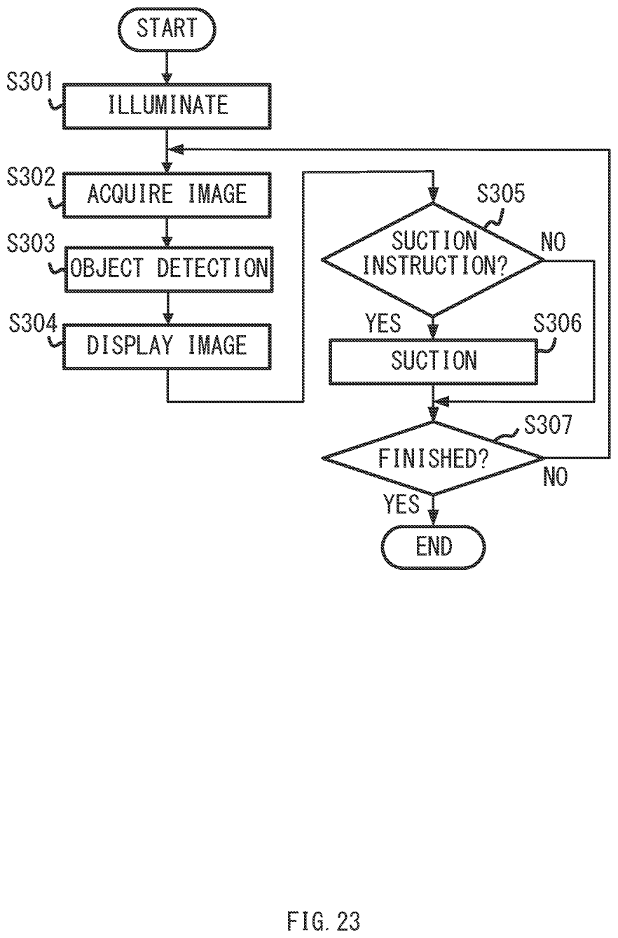

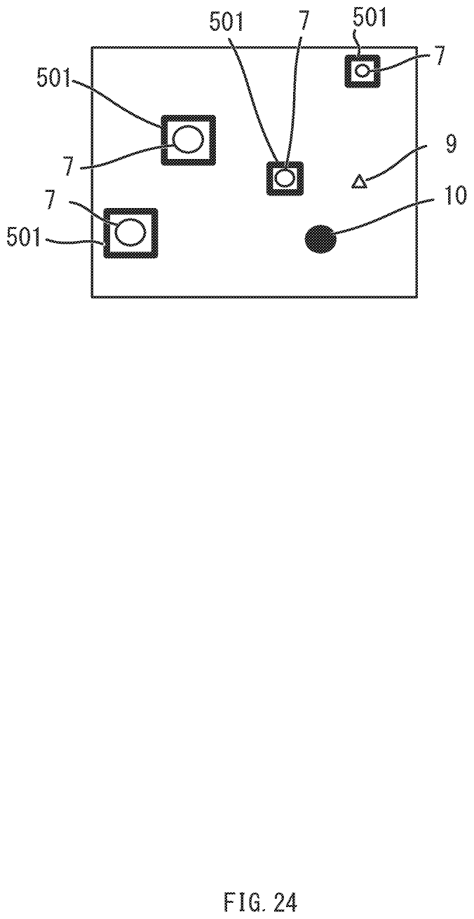

FIG. 23 is a flowchart illustrating an example of cell recovery processing to be performed by a cell recovery apparatus according to the present embodiment. FIG. 24 is a diagram illustrating another example of an image to be displayed on a display apparatus 133. The cell recovery apparatus according to the present embodiment is an apparatus that automatically sorts cells cultured in suspension culture.

An apparatus configuration of the cell recovery apparatus according to the present embodiment is similar to the cell recovery apparatus according to the fifth embodiment, and is similar to the cell recovery apparatus 100 illustrated in FIG. 2 except that a control apparatus 130 controls a recovery apparatus 120 in response to an instruction from a user. The cell recovery apparatus according to the present embodiment differs from the cell recovery apparatus according to the fifth embodiment in that the control apparatus 130 performs object detection and the user is notified of the presence of the cells in a predetermined region 143 based on a result of the object detection.

The cell recovery processing illustrated in FIG. 23 is started in response to a start instruction from the user, for example, when a processor in the control apparatus 130 executes a cell recovery program. When the cell recovery processing is started, the cell recovery apparatus first illuminates the predetermined region 143 (step S301), acquires an image of the predetermined region 143 (step S302), and performs object detection for the acquired image (step S303). The processing in step S301 to step S303 is similar to the processing in step S10 to step S30 illustrated in FIG. 4.

Then, the cell recovery apparatus displays the acquired image (step S304). The control apparatus 130 causes the display apparatus 133 to display the acquired image, together with auxiliary information based on a result of the object detection, as illustrated in FIG. 24, for example. Specifically, the control apparatus 130 displays a bounding box 501 surrounding a cell 7 to be recovered on an image to visually notify the user of the presence of the cell to be recovered. The user sees the image displayed on the display apparatus 133, to determine whether or not the cell 7 to be recovered exists in a recovery region. A cell 9 and a cell 10 illustrated in FIG. 24 are respectively cells other than the cell to be recovered.

Then, the cell recovery apparatus determines the presence or absence of a suction instruction (step S305). When the user sees the image to confirm the presence of the cell to be recovered, and presses a suction button provided in an operation section 112, for example, the control apparatus 130 detects a suction instruction (YES in step S305), and performs suction control processing (step S306). The processing in step S306 is similar to the processing in step S40 illustrated in FIG. 4.

When the suction control processing ends, the cell recovery apparatus determines whether or not the cell recovery processing ends (step S307). The cell recovery apparatus repeatedly performs the processing in step S302 to step S306 until it is determined in step S307 that the cell recovery processing ends, and finishes the cell recovery processing when it is determined in step S307 that the cell recovery processing ends.

The cell recovery apparatus according to the present embodiment also makes it possible to sort the cells while suppressing a damage to the cells, like the cell recovery apparatus according to the fifth embodiment. The cell recovery apparatus according to the present embodiment makes it possible to efficiently sort the cells because information for assisting the user in specifying the cell to be recovered is displayed on the image.

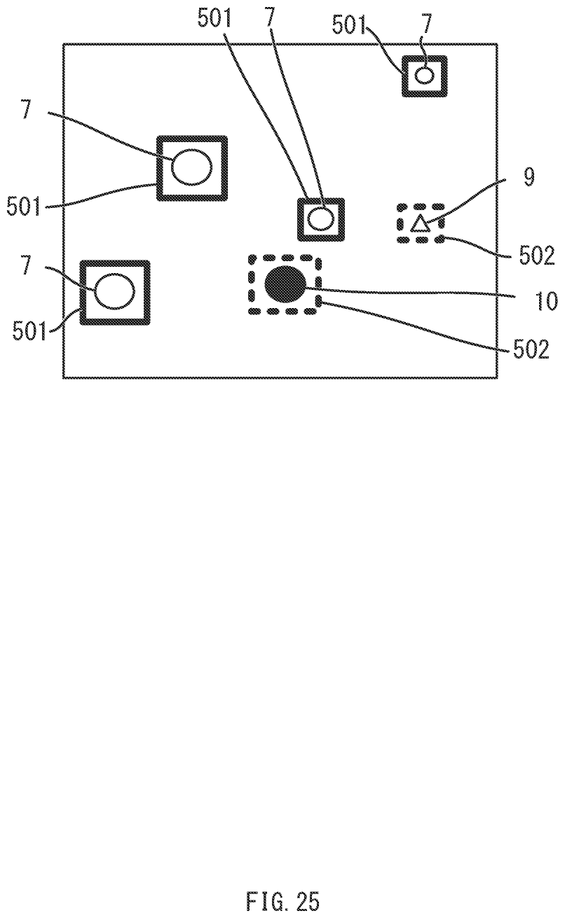

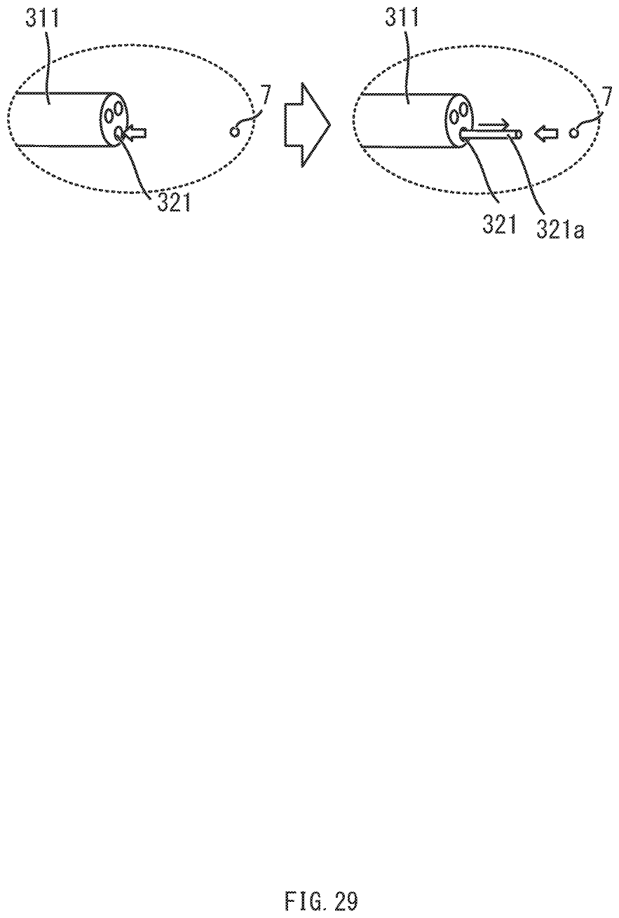

In FIG. 25 to FIG. 28 are diagrams each illustrating still another example of the image to be displayed on the display apparatus 133. FIG. 29 is a diagram for describing an operation of an extension nozzle 321a. Although an example in which the cell 7 to be recovered is displayed by being surrounded by the bounding box 501 has been illustrated in the above-described embodiment, auxiliary information to be displayed on the image is not limited to the bounding box 501 surrounding the cell 7 to be recovered.

As illustrated in FIG. 25, for example, bounding boxes 502 respectively surrounding the cells (the cell 9 and the cell 10) not to be recovered may be displayed. The bounding box 502 may be displayed in a different color, for example, such that it can be distinguished at a glance from the bounding box 501. When the bounding box 502 is displayed, the user can be notified of the presence of the cells not to be recovered. Accordingly, the cells not to be recovered can be avoided being erroneously recovered.

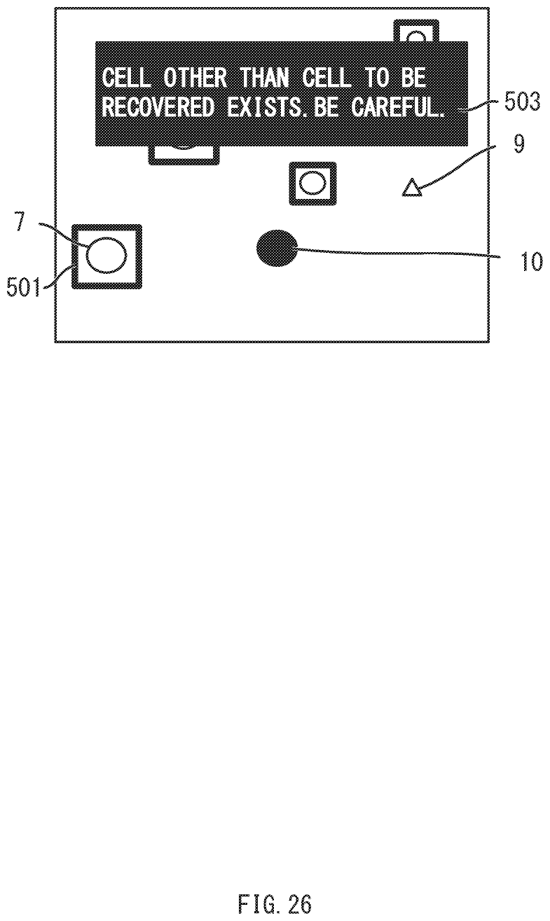

When the presence of the cells (the cell 9 and the cell 10) not to be recovered has been detected, as illustrated in FIG. 26, a message 503 including information about that may be displayed. In this case, the user can also be notified of the presence of the cells not to be recovered, like when the bounding box 502 is displayed. Accordingly, the cells not to be recovered can be avoided being erroneously recovered.

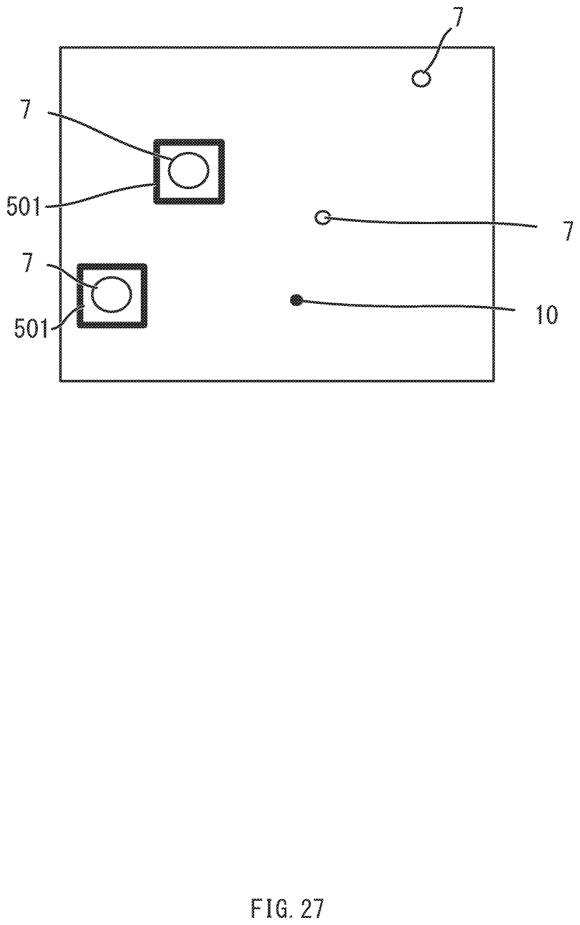

As illustrated in FIG. 27, only the cell 7 to be recovered existing in the recovery region may be surrounded by the bounding box 501 instead of all the cells 7 to be recovered on the image being respectively surrounded by the bounding boxes 501. That is, the cell 7 that cannot be recovered because it is too farther away from the recovery nozzle 121 may not be surrounded by the bounding box 501 even when detected by object detection. As a result, the presence of the cell 7 that cannot be recovered can be avoided being notified. Only the cell not to be recovered existing in the recovery region may be surrounded by the bounding box 502 instead of all the cells not to be recovered on the image being respectively surrounded by the bounding boxes 502. Particularly in an apparatus configuration capable of measuring a three-dimensional position of the cell, a distance of the cell from the recovery nozzle 121 can be measured. Accordingly, recoverability may be visualized from the size, the thickness, the color, and the like of the bounding box. For example, a blue frame and a red frame, for example, may be respectively used for the cell that is far from the recovery nozzle 121 and is less likely to be recovered and the cell that is close to the recovery nozzle 121 and is likely to be recovered.

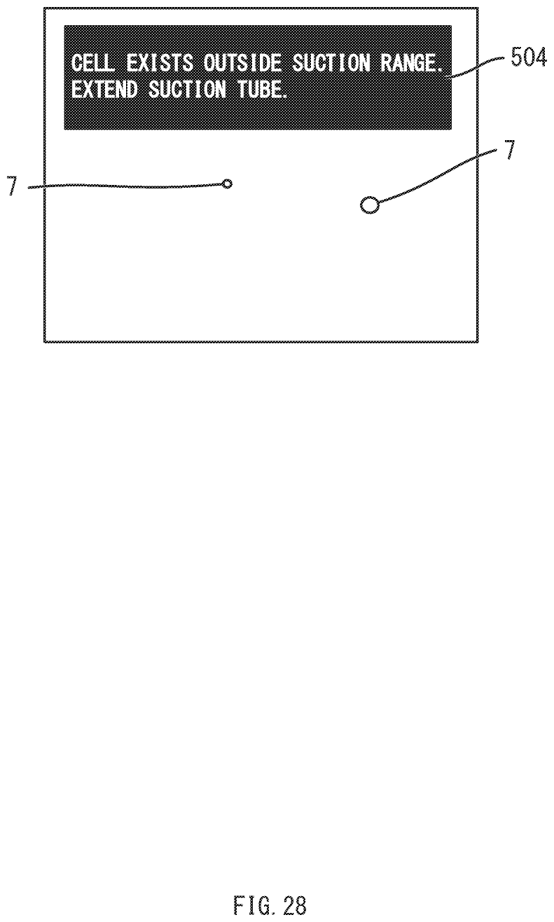

When the cell 7 to be recovered existing outside the recovery region has been detected, as illustrated in FIG. 28, a message 504 including information about an operation for recovering the cell may be displayed. If the cell recovery apparatus 300 is used, the user may operate an operation section 312 to move an insertion section 311 such that the cell enters the recovery region and recover the cell. The user may operate the operation section 312 to extend the extension nozzle 321a from the recovery nozzle 321 and recover the cell as illustrated in FIG. 29.

### Seventh Embodiment

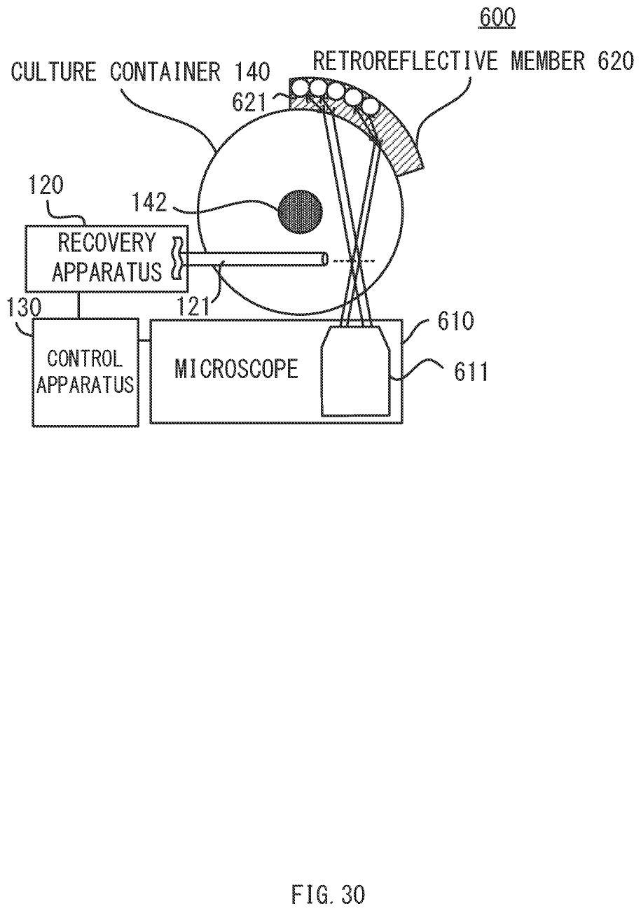

FIG. 30 is a diagram illustrating a configuration of a cell recovery apparatus 600 according to the present embodiment. The cell recovery apparatus 600 is an apparatus that automatically sorts cells cultured in suspension culture. The cell recovery apparatus 600 is similar to the cell recovery apparatus 100 in that it includes a recovery apparatus 120 and a control apparatus 130.

The cell recovery apparatus 600 differs from the cell recovery apparatus 100 in that it includes a microscope 610 as an example of an observation apparatus and a retroreflective member 620 to be attached to a container 140 instead of the observation apparatus 110. The microscope 610 is an apparatus for observing the inside of the container 140 from outside the container 140, and the objective lens 611 is arranged toward a side surface of the container 140, as illustrated in FIG. 30, for example. The microscope 610 may allow observation of an object using a phase difference observation method, or may allow observation of an object using a bright field observation method, for example.

The retroreflective member 620 is used to cancel a lens effect of receiving light emitted from the microscope 610 on the side surface of the container 140. The retroreflective member 620 includes an array having many small reflective elements 621 arranged therein in a horizontal direction. An example of the reflective elements 621 is a prism or spherical glass beads. The retroreflective member 620 reflects incident light on the reflective elements 621, and makes the incident light travel in the same optical path as and on the opposite direction to when incident.

When the inside of the container 140 is observed from outside the container 140, an observation performance generally depends on a shape of the container 140. However, when the microscope 610, together with the retroreflective member 620, is used, a lens effect to be produced on a side surface of the container 140 can be canceled, as described in International Publication No. 2019/163167, for example. Therefore, the inside of the container 140 can be observed in a stabler performance than that in the conventional technique from outside the container 140.

The cell recovery apparatus 600 according to the present embodiment also makes it possible to sort cells efficiently and while suppressing a damage to the cells without imposing a burden of recovery work on a user.

### Eighth Embodiment

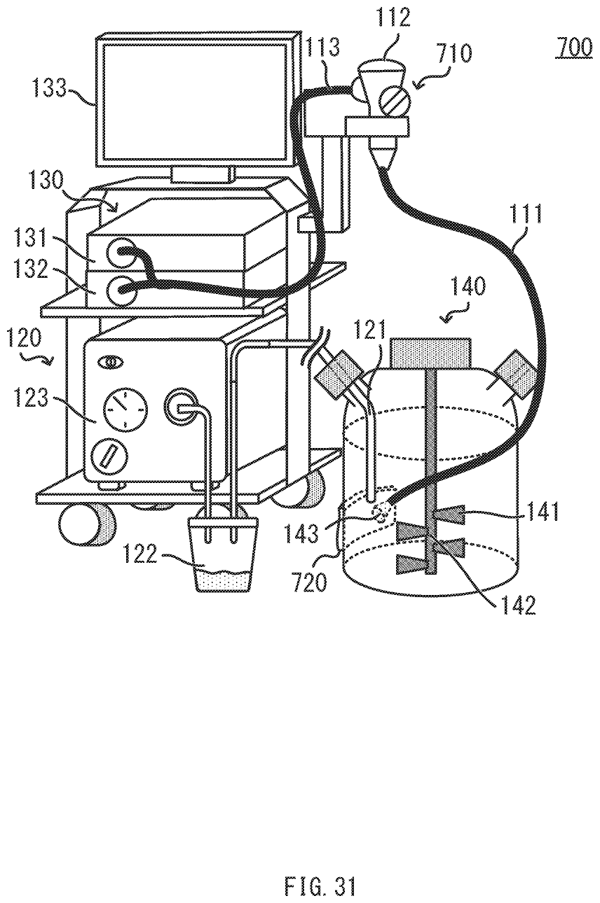

FIG. 31 is a diagram illustrating a configuration of a cell recovery apparatus 700 according to an eighth embodiment. The cell recovery apparatus 700 is an apparatus that automatically sorts cells cultured in suspension culture. The cell recovery apparatus 700 differs from the cell recovery apparatus 600 in that it includes an observation apparatus 710 instead of the microscope 610. The observation apparatus 710 is similar to the observation apparatus 110 included in the cell recovery apparatus 100 according to the first embodiment but differs from the observation apparatus 110 in that it is used to observe the inside of a container 140 from outside the container 140 in combination with a retroreflective member 720.

The cell recovery apparatus 700 according to the present embodiment also makes it possible to observe the inside of the container 140 in a stabler performance than that in the conventional technique from outside the container 140, like the cell recovery apparatus 600. Therefore, the cell recovery apparatus 700 makes it possible to sort the cells efficiently and while suppressing a damage to the cells without imposing a burden of recovery work on a user, like the cell recovery apparatus 600.

The above-described embodiments respectively represent specific examples to make understanding of the invention easy, and the present invention is not limited to the embodiments. Modifications to the above-described embodiments and alternatives to the above-described embodiments can be encompassed by the invention. That is, in each of the embodiments, constituent elements can be deformed without departing from the spirit and scope of the invention. A plurality of constituent elements disclosed in one or more of the embodiments can be appropriately combined to implement a new embodiment. Some of the constituent elements illustrated in each of the embodiments may be deleted, or some constituent elements may be added to the constituent elements illustrated in the embodiment. Further, a processing procedure illustrated in each of the embodiments may be performed by replacing orders, unless otherwise contradicted. That is, a cell recovery apparatus, a cell recovery method, and a computer readable medium according to the present invention can be subjected to various modifications and variations without departing from the scope of the claims.

Although an example in which a recovery container is placed outside a suction device is illustrated in the above-described embodiments, for example, the recovery container may be contained in the suction device. Although an example in which a control apparatus is connected to an observation apparatus and a recovery apparatus via a wired cable is illustrated in the above-described embodiments, communication between the apparatuses may be performed by wireless communication, or communication between the apparatuses may be performed with another apparatus such as an access point interposed therebetween, for example. Although an example in which a control apparatus is arranged spatially in close proximity to an observation apparatus, a recovery apparatus, and a container is illustrated in the above-described embodiments, the control apparatus may be placed in a cloud environment or may be connected to another apparatus via the Internet, for example, as long as a communication delay does not affect recovery processing.

Although an example in which a cell recovery apparatus is used to sort cells is illustrated in the above-described embodiments, the cell recovery apparatus may be used to merely recover cells from a container, or the recovered cells may be selected after that.

Although an example in which a cell recovery apparatus allows observation of a culture medium from inside or from outside a container is illustrated in the above-described embodiments, the cell recovery apparatus may allow observation of the culture medium from both inside and outside the container. At this time, an observation apparatus may include an apparatus for observing the culture medium from inside the container and an apparatus for observing the culture medium from outside the container.

Although an example in which a position of a cell is detected to use the position of the cell for control of a recovery operation is illustrated in the above-described embodiments, a movement speed of the cell may be detected in addition to the position of the cell. The control apparatus 130 may use the position and the movement speed of the cell for control of the recovery operation.

What is claimed is:

1. A cell recovery apparatus comprising:
an observation apparatus includes an insertion section to be inserted into a medium in a container, the observation apparatus being for observing the inside of the medium via the insertion section; and
a recovery apparatus that is positioned relative to a predetermined region to be observed by the observation apparatus and recovers a cell in the medium from the container.

2. The cell recovery apparatus according to claim 1, wherein
the recovery apparatus
includes a channel provided in the insertion section, and
recovers the cell in the medium from the container via the channel.

3. The cell recovery apparatus according to claim 1, wherein
the recovery apparatus includes
a second insertion section to be inserted into the medium in the container, and
a channel provided in the second insertion section, and
recovers the cell in the medium from the container via the channel.

4. The cell recovery apparatus according to claim 3, further comprising
a bundle section that bundles the insertion section and the second insertion section, or a housing that accommodates the insertion section and the second insertion section.

5. The cell recovery apparatus according to claim 2, wherein
the recovery apparatus
further includes a suction section that generates a suction force, and
sucks the cell in the medium from the container with the suction force generated by the suction section, and outputs the sucked cell to a recovery container via the channel.

6. The cell recovery apparatus according to claim 1, wherein
the recovery apparatus further includes a partition section that partitions a region in the medium in which the cell is to be recovered by the recovery apparatus.

7. The cell recovery apparatus according to claim 1, wherein
the observation apparatus further includes
an illumination section that is provided in the insertion section and irradiates the predetermined region with illumination light emitted from a light source,
an imaging optical system that is provided in the insertion section and forms an optical image of the predetermined region, and
a light receiving section that is provided in the insertion section and receives the optical image.

8. The cell recovery apparatus according to claim 7, wherein
the imaging optical system includes a stereo optical system.

9. The cell recovery apparatus according to claim 1, further comprising
a control apparatus, wherein
the observation apparatus includes an imaging apparatus that images the predetermined region, and
the control apparatus controls the recovery apparatus based on a result of analysis for the image of the predetermined region acquired using the imaging apparatus.

10. The cell recovery apparatus according to claim 9, wherein
the control apparatus controls the recovery apparatus based on a result of object detection for the image of the predetermined region.

11. The cell recovery apparatus according to claim 10, wherein
the control apparatus
causes the recovery apparatus to perform a recovery operation when a cell to be recovered is detected by the object detection, and
causes the recovery apparatus not to perform the recovery operation when the cell to be recovered is not detected by the object detection.

12. The cell recovery apparatus according to claim 10, wherein
the control apparatus
causes the recovery apparatus to perform a recovery operation when a cell to be recovered is detected and a cell not to be recovered is not detected by the object detection,
causes the recovery apparatus not to perform the recovery operation when the cell to be recovered is not detected by the object detection, and
causes the recovery apparatus not to perform the recovery operation when the cell not to be recovered is detected by the object detection.

13. The cell recovery apparatus according to claim 11, wherein
the control apparatus causes the recovery apparatus to perform the recovery operation using a suction force corresponding to a position of the cell to be recovered.

14. The cell recovery apparatus according to claim 1, further comprising
a control apparatus, wherein
the observation apparatus includes an imaging apparatus that images the predetermined region, and
the control apparatus causes a display section to display the image of the predetermined region acquired using the imaging apparatus.

15. The cell recovery apparatus according to claim 14, wherein
the control apparatus notifies the presence of a cell in the predetermined region based on a result of object detection for an image of the predetermined region.

16. A cell recovery method comprising:
imaging a predetermined region in a medium in a container with an insertion section in an imaging apparatus inserted into the medium; and
recovering a cell in the medium from the container by a recovery apparatus positioned relative to the predetermined region in response to a result of object detection for an image acquired by the imaging apparatus.

17. A non-transitory computer readable medium storing a program for causing a computer to perform processing for:
performing object detection for an image acquired by an imaging apparatus imaging a predetermined region in a medium in a container with an insertion section in the imaging apparatus inserted into the medium; and
controlling a recovery apparatus that is positioned relative to the predetermined region and recovers a cell in the medium from the container in response to a result of the object detection.
